(19) United States
(12) Patent Application Publication
(10) Pub. No.: US 2011/0212091 A1
(43) Pub. Date: Sep. 1, 2011

(54) MATERIALS AND METHODS FOR INHIBITING CANCER CELL INVASION

(75) Inventors: Kari Alitalo, Helsinki (FI); Kaisa Lehti, Espoo (FI); Jorma Keski-Oja, Helsinki (FI); Annamari Alitalo, Helsinki (FI)

(73) Assignee: LICENTIA LTD., HELSINKI (FI)

(21) Appl. No.: 13/061,058

(22) PCT Filed: Sep. 2, 2009
(86) PCT No.: PCT/FI09/50697

§ 371 Date: May 20, 2011

(30) Foreign Application Priority Data
Sep. 3, 2008 (US) 61/093925
Mar. 2, 2009 (US) 61/156634

(51) Int. Cl. A61K 39/395; C12N 5/12; G01N 33/566; C07K 16/40; A61K 39/00; A61P 35/00; A61P 37/04

(52) U.S. Cl. 424/133.1; 435/334; 435/7.23; 530/388.22; 530/387.9; 530/387.3; 424/139.1; 530/391.7; 424/143.1; 424/185.1; 424/184.1

(57) ABSTRACT

The invention provides an isolated antibody or antibody fragment thereof that binds an extracellular epitope of a fibroblast growth factor receptor-4 (FGFR4) that is expressed by mammalian cells and inhibits cancer cell invasion. Optionally, the antibody or fragment thereof binds an epitope of FGFR4 that is bound by monoclonal antibody F90-10C5, or comprises complementarity determining regions identical to those of monoclonal antibody F90-10C5. Also provided are methods of using the antibody or fragment thereof to modulate invasion, ingrowth, or metastasis of cancer cells and treat cancer in a subject. The invention additionally provides a method of identifying an antibody or antibody fragment that inhibits invasiveness.

### CROSS-REFERENCE TO RELATED APPLICATIONS

This application claims priority to U.S. Provisional Patent Application No. 61/093,925, filed Sep. 3, 2008, and U.S. Provisional Patent Application No. 61/156,634, filed Mar. 2, 2009.

### TECHNICAL FIELD OF THE INVENTION

The invention generally relates to cancer therapy and also to antibodies and antibody fragments that bind fibroblast growth factor receptor-4 (FGFR4) and uses thereof and combination therapies containing them.

### BACKGROUND OF THE INVENTION

Tumor cell invasion plays an important role in cancer pathogenesis. The invasion of carcinoma cells through underlying basement membrane and metastasis to distant organs are considered rate limiting steps in carcinogenesis and cancer spread. During these processes, tumor cells rely on targeted proteolytic activities on the cell surface to traverse basement membrane barriers, and invade and grow in collagen or fibrin-rich interstitial and temporary matrixes. The formation of secondary tumors in distant regions of the body complicates therapeutic options and often results poor clinical outcomes in cancer patients.

Current strategies in cancer treatment focus on blocking tumor growth through pro-apoptotic, anti-proliferative, and anti-angiogenic therapies. For example, growth factor receptors, e.g., fibroblast growth factor receptors, have been suggested as a possible target for inhibiting proliferation. See, e.g., St. Bernard et al., Endocrinology, 146(3): 1145-1153 (2005). Possible regulators of growth factor receptor signaling include, e.g., small molecules, inactivating ligands, and antibodies. Kwabi-Addo et al., Endocrine-Related Cancer, 11: 709-724 (2004). Chen et al., Hybridoma, 24(3): 152-159 (2005), for instance, purportedly identified antibodies that bind FGFR4 on human breast cancer tumor cell lines. However, attempts to inhibit tumor invasion have not been as successful. Thus, the development of novel interventions to target cell invasion is of key importance for more efficient cancer treatments.

### SUMMARY OF THE INVENTION

The invention generally relates to materials that are useful in the treatment of neoplastic disorders such as cancer, including antibody substances, nucleic acids, polypeptides, and compositions. The invention further relates to methods of using such materials, including methods of treatment, medical uses, and uses for making pharmaceutical compositions. The invention also relates to tools for screening for novel therapeutics and new combination therapies.

The invention provides an isolated antibody or antibody fragment that (i) binds an extracellular epitope of a fibroblast growth factor receptor-4 (FGFR4) that is expressed by mammalian cells and (ii) inhibits cancer cell invasion. In addition, the invention provides monoclonal antibody F90-10C5, as well as an isolated antibody, antibody fragment, or polypeptide that comprises one or more, and preferably all complementarity determine regions (CDR) of monoclonal antibody F90-10C5 and binds an extracellular epitope of FGFR4 that is expressed in mammalian cells. In another aspect, the invention provides an isolated antibody, such as a monoclonal antibody, or fragment thereof that binds an epitope of FGFR4 that is bound by monoclonal antibody F90-10C5. The FGFR4 recognized by the antibody can comprise the amino acid sequence of the FGFR4 G388 protein or the FGFR4 R388 variant. Compositions comprising the antibody, fragment thereof, or polypeptide, optionally combined with other therapeutics and/or with pharmaceutically acceptable carrier(s), excipient(s), adjuvants, or the like, also are included in the invention.

The invention also provides materials and methods for making the claimed antibody or fragment thereof. For example, the invention provides an isolated polynucleotide that encodes the inventive antibody or fragment thereof, a vector comprising the polynucleotide, an isolated host cell comprising the polynucleotide or vector, and a hybridoma. An isolated polynucleotide comprising a nucleotide sequence that encodes at least one amino acid sequence selected from the group consisting of an antibody heavy chain variable region and an antibody light chain variable region also is provided by the invention. The heavy chain variable region and light chain variable region comprise complementarity determine regions (CDR) identical to monoclonal antibody F90-10C5 CDRs. The invention also provides a method of identifying an antibody or antibody fragment. The method comprises obtaining one or more antibodies or antibody fragments that bind FGFR4; screening the antibodies or antibody fragments in a tumor cell invasiveness assay; and identifying an antibody that inhibits invasiveness in the assay by at least 50%.

The invention further includes methods of using the inventive antibody or fragment thereof. For example, a method of modulating invasion, ingrowth, or metastasis of cancer cells is provided. The method comprises contacting a population of cancer cells with a composition comprising the inventive antibody or fragment thereof in an amount effective to modulate cancer cell invasion, ingrowth, or metastasis. The method can be performed in vivo, such that the cancer cells are in a mammalian subject, and the contacting step comprises administering the composition to the mammalian subject.

In another aspect, the invention includes a method of treating a subject by administering a composition comprising the inventive antibody, fragment thereof, or polypeptide. For example, in one embodiment, the method comprises selecting for treatment a mammalian subject diagnosed with or treated for cancer; and administering to the subject the inventive composition in an amount effective to modulate cancer cell invasion, ingrowth, or metastasis. A method of treating cancer also is provided. The method comprises administering to the subject the composition comprising the inventive antibody or fragment thereof in an amount effective to treat cancer. Optionally, (i) the antibody or fragment thereof binds an epitope of FGFR4 that is bound by monoclonal antibody F90-10C5 and (ii) the method further comprises contacting the population of cancer cells with (or administering to the subject) an antibody or fragment thereof that binds an epitope of FGFR4 that is different than the epitope recognized by mAb F90-10C5. Alternatively or in addition, the method can comprise contacting the population of cancer cells with (or administering to the subject) an MT1-MMP inhibitor.

In some variations of the invention, the subject has a cancer that includes cells that contain at least one FGFR4 allele that is characterized by an arginine at amino acid position 388 (FGFR4 R388). This particular allele is linked to increased cancer cell invasion and poor patient prognosis, and may obtain unexpected benefit from methods of the invention. The cancer cells may have an FGFR4 R288 allele due to mutation localized to the cancer or due to inheritance of the allele. Selecting for treatment a cancer patient with one or more FGFR4 R388 alleles in the cancer is specifically contemplated as an aspect of the invention.

The following numbered paragraphs each succinctly define one or more exemplary variations of the invention:

1. An isolated antibody or antibody fragment thereof that binds an extracellular epitope of a fibroblast growth factor receptor-4 (FGFR4) that is expressed by mammalian cells and inhibits cancer cell invasion.

2. An isolated polypeptide that comprises a fragment of an antibody that binds an extracellular epitope of a fibroblast growth factor receptor-4 (FGFR4) that is expressed by mammalian cells, wherein the antibody and the polypeptide inhibit cancer cell invasion.

3. An isolated antibody or antibody fragment thereof that bind an extracellular epitope of a fibroblast growth factor receptor-4 (FGFR4) on mammalian cells that express FGFR4 R388 (SEQ ID NO: 2) and inhibits fibroblast growth factor 2 (FGF2)-induced phosphorylation of FGFR4 in the cells.

4. An isolated polypeptide that comprises a fragment of an antibody that bind an extracellular epitope of a fibroblast growth factor receptor-4 (FGFR4) on mammalian cells that express FGFR4 R388 (SEQ ID NO: 2), wherein the antibody and the polypeptide inhibit fibroblast growth factor 2 (FGF2)-induced phosphorylation of FGFR4 in the cells.

5. An isolated antibody or antibody fragment thereof that bind an extracellular epitope of a fibroblast growth factor receptor-4 (FGFR4) on mammalian cells that co-express FGFR4 R388 (SEQ ID NO: 2) and fibroblast growth factor receptor-1 (FGFR1), wherein the antibody or fragment enhances fibroblast growth factor 2 (FGF2)-induced FGFR1 degradation in the cells.

6. An isolated polypeptide that comprises a fragment of an antibody that bind an extracellular epitope of a fibroblast growth factor receptor-4 (FGFR4) on mammalian cells that co-express FGFR4 R388 (SEQ ID NO: 2) and fibroblast growth factor receptor-1 (FGFR1), wherein the antibody and the polypeptide enhance fibroblast growth factor 2 (FGF2)-induced FGFR1 degradation in the cells.

7. The antibody, antibody fragment, or polypeptide of paragraph 5 or 6 that also inhibits FGF2-induced phosphorylation of FGFR4 in the cells.

8. An isolated antibody or antibody fragment thereof that bind an extracellular epitope of a fibroblast growth factor receptor-4 (FGFR4) on mammalian cells that co-express FGFR4 R388 (SEQ ID NO: 2) and membrane type-1 metalloproteinase (MT1-MMP), wherein the antibody or fragment inhibits complex formation between FGFR4 and MT1-MMP in the cells.

9. An isolated polypeptide that comprises a fragment of an antibody that binds an extracellular epitope of a fibroblast growth factor receptor-4 (FGFR4) on mammalian cells that co-express FGFR4 R388 (SEQ ID NO: 2) and membrane type-1 metalloproteinase (MT1-MMP), wherein the antibody and the polypeptide inhibit complex formation between FGFR4 and MT1-MMPin the cells.

10. The antibody, antibody fragment, or polypeptide of any one of paragraphs 3-9 that inhibits cancer cell invasion.

11. The antibody, antibody fragment, or polypeptide of any one of paragraphs 1-10, wherein the antibody binds an FGFR4 that comprises the amino acid sequence of SEQ ID NO: 1.

12. The antibody, antibody fragment, or polypeptide of any one of paragraphs 1-10, wherein the antibody binds an FGFR4 that comprises the amino acid sequence of SEQ ID NO: 2.

13. The antibody, antibody fragment, or polypeptide of any one of paragraphs 1-12, wherein the antibody binds an FGFR4 peptide that consists of an amino acid sequence selected from the group consisting of SEQ ID NOS: 5-9.

14. The antibody, antibody fragment, or polypeptide of paragraph 13 that binds an FGFR4 peptide consisting of SEQ ID NO: 7.

15. The antibody, antibody fragment, or polypeptide of paragraph 13, wherein the antibody or antibody fragment binds an epitope of SEQ ID NO: 1 or 2 that comprises amino acid residues 79-81.

16. An isolated antibody or fragment thereof that binds an epitope of FGFR4 that is bound by monoclonal antibody F90-10C5.

17. The polypeptide of any one of paragraphs 2, 4, 6, and 9, wherein the antibody is monoclonal antibody F90-10C5.

18. The antibody fragment or polypeptide of any one of paragraphs 1-17, wherein the antibody fragment is an ScFv, Fv, Fab′, Fab, diabody, or F(ab′)2 antigen-binding fragment of an antibody.

19. An isolated antibody, antibody fragment, or polypeptide that comprises all complementarity determine regions (CDR) of monoclonal antibody F90-10C5, wherein the antibody, antibody fragment, or polypeptide binds an extracellular epitope of FGFR4 that is expressed by mammalian cells.

20. The antibody, antibody fragment, or polypeptide of paragraph 19 that comprises the variable regions of monoclonal antibody F90-10C5.

21. The antibody, antibody fragment, or polypeptide of any one of paragraphs 1-20, wherein the antibody or antibody fragment inhibits invasion of MDA-MB-231 cells expressing FGFR4 R388 protein in a tumor cell invasiveness assay.

22. The antibody, antibody fragment, or polypeptide of paragraph 21, wherein the antibody or antibody fragment reduces cell invasion in a tumor cell invasiveness assay by at least 25%.

23. The antibody, antibody fragment, or polypeptide of paragraph 21, wherein the antibody or antibody fragment reduces cell invasion in a tumor cell invasiveness assay by at least 50%.

24. The antibody of paragraph 20 that is monoclonal antibody F90-10C5.

25. An isolated antibody, antibody fragment, or polypeptide that comprises all complementarity determine regions (CDR) of monoclonal antibody F85-6C5, wherein the antibody, antibody fragment, or polypeptide binds an extracellular epitope of FGFR4 that is expressed by mammalian cells.

26. The antibody, antibody fragment, or polypeptide of paragraph 25 that comprises the variable regions of monoclonal antibody F85-6C5.

27. The antibody of paragraph 26 that is monoclonal antibody F85-6C5.

28. An isolated antibody, antibody fragment, or polypeptide that comprises all complementarity determine regions (CDR) of monoclonal antibody F90-3B6, wherein the antibody, antibody fragment, or polypeptide binds an extracellular epitope of FGFR4 that is expressed by mammalian cells.

29. The antibody, antibody fragment, or polypeptide of paragraph 28 that comprises the variable regions of monoclonal antibody F90-3B6.

30. The antibody of paragraph 29 that is monoclonal antibody F90-3B6.

31. The antibody or antibody fragment of any one of paragraphs 1, 3, 5, 7, 8, 10-15, and 21-23 wherein the antibody is a monoclonal antibody.

32. The antibody or antibody fragment of any one of paragraphs 1, 3, 5, 7, 8, 10-15, and 20-31, wherein the antibody is a humanized antibody, a human antibody, or a chimeric antibody.

33. A humanized antibody that comprises the variable regions of mAb F90-10C5, F85-6C5, or F90-3B6 or a fragment of any of the foregoing that binds FGFR4.

34. The antibody, antibody fragment, or polypeptide of any one of paragraphs 1-33, further comprising an anti-neoplastic or cytotoxic agent conjugated or attached thereto.

35. The antibody, antibody fragment, or polypeptide of paragraph 34, wherein the anti-neoplastic agent comprises a radionucleotide.

36. A composition comprising the antibody, antibody fragment, or polypeptide of any one of paragraphs 1-35 and a physiologically acceptable carrier.

37. The composition of paragraph 36, further comprising a standard of care anti-cancer therapeutic compound.

38. The composition of paragraph 36 or 37, further comprising an agent that inhibits VEGF-D or VEGF-C stimulation of VEGFR-3 or VEGFR-2.

39. The composition of paragraph 38, wherein the agent comprises a member selected from the group consisting of:

antibodies or antibody fragments that bind to VEGF-C, VEGF-D, or the extracellular domain of VEGFR-3 or VEGFR-2;

a soluble protein comprising a VEGFR-3 extracellular domain or fragment thereof effective to bind VEGF-C or VEGF-D; and a soluble protein comprising a VEGFR-2 extracellular domain or fragment thereof effective to bind VEGF-C or VEGF-D.

40. The composition of any one of paragraphs 36-38, wherein the antibody, antibody fragment, or polypeptide is a monoclonal antibody or fragment thereof (“the first monoclonal antibody or fragment thereof”).

41. The composition of paragraph 40, further comprising a second monoclonal antibody or fragment thereof that binds a second epitope of FGFR4 that is different than the epitope recognized by the first monoclonal antibody or fragment thereof.

42. The composition of paragraph 41, wherein the second monoclonal antibody or fragment thereof is a human or humanized antibody.

43. The composition of any one of paragraphs 36-42, further comprising a membrane type-1 metalloproteinase (MT1-MMP) inhibitor.

44. The composition of paragraph 43, wherein the MT1-MMP inhibitor is an antibody or fragment thereof that binds MT1-MMP or a small molecule inhibitor of MT1-MMP.

45. The composition of paragraph 43, wherein the MT1-MMP inhibitor is an inhibitor nucleic acid that hybridizes with MT 1-MMP genomic DNA or mRNA and inhibits MT 1-MMP transcription or translation.

46. An isolated polynucleotide that comprises a nucleotide sequence that encodes the antibody, antibody fragment, or polypeptide of any one of paragraphs 1-33.

47. A vector that comprises the polynucleotide of paragraph 46.

48. The vector of paragraph 47 that is an expression vector.

49. The vector of paragraph 48 that is a replication-deficient viral vector.

50. A composition comprising the vector of paragraph 49 and a physiologically acceptable carrier.

51. An isolated cell transformed or transfected with the polynucleotide or vector of any one of paragraphs 46-49.

52. An isolated cell that produces the antibody, antibody fragment, or polypeptide of any one of paragraphs 1-33.

53. A hybridoma that produces the monoclonal antibody or antibody fragment of any one of paragraphs 24, 27, and 30-32.

54. A method of modulating invasion, ingrowth, or metastasis of cancer cells, wherein the method comprises contacting a population of cancer cells with an antibody, antibody fragment, polypeptide, polynucleotide, or composition of any one of paragraphs 1-50, in an amount effective to modulate cancer cell invasion, ingrowth, or metastasis.

55. The method of paragraph 54, wherein the cancer cells are in a mammalian subject, and the contacting comprises administering the composition to the mammalian subject.

56. A method of treating a mammalian subject comprising:

selecting for treatment a mammalian subject diagnosed with or treated for cancer; and administering to the subject the composition of any one of paragraphs 36-45 and 50, in an amount effective to modulate cancer cell invasion, ingrowth, or metastasis.

57. The method of paragraph 55 or 56, wherein (i) the composition comprises an antibody, antibody fragment, or polypeptide according to any one of paragraphs 13-17, and wherein the method further comprises administering to the mammalian subject an antibody or fragment thereof that binds a second epitope of FGFR4 that is different than the epitope recognized by the antibody, antibody fragment, or polypeptide of the composition.

58. The method of paragraph 57, wherein the antibody or fragment thereof that binds a second epitope is mAb F90-3B6 or a fragment thereof.

59. The method of paragraph 55 or 56, wherein the composition is a composition according to any one of paragraphs 36-42, and wherein the method further comprises administering to the mammalian subject a composition comprising a membrane type-1 metalloproteinase (MT1-MMP)inhibitor.

60. The method of paragraph 59, wherein the MT1-MMP inhibitor is an antibody or fragment thereof that binds MT1-MMP or a small molecule inhibitor of MT1-MMP.

61. The method of paragraph 55 or 56, wherein the composition is a composition according to any one of paragraphs 36-37 and 40-42, and wherein the method further comprises administering to the mammalian subject a composition that comprises an agent that inhibits VEGF-D or VEGF-C stimulation of VEGR-3 or VEGFR-2.

62. The method of any one of paragraphs 55-61, wherein the method further comprises administering to the mammalian subject a standard of care anti-cancer therapy.

63. A method of treating cancer in a subject, wherein the method comprises administering to the subject the composition of any one of paragraphs 36-45 and 50, in an amount effective to treat cancer.

64. Use of an antibody, antibody fragment, polypeptide, polynucleotide, or composition of any one of paragraphs 1-50 to inhibit invasion, ingrowth, or metastasis of cancer cells in a mammalian subject.

65. The use according to paragraph 64 in combination with a MT 1-MMP inhibitor or an inhibitor of VEGF-C or VEGF-D binding to VEGFR-3 or VEGFR-2 to inhibit invasion, ingrowth, or metastasis of cancer cells in a mammalian subject.

66. The use according to paragraph 64 or 65, wherein (i) the composition comprises an antibody, antibody fragment, or polypeptide according to any one of paragraphs 13-17, used in combination with an antibody or fragment thereof that binds a second epitope of FGFR4 that is different than the epitope recognized by the antibody, antibody fragment, or polypeptide of the composition.

67. The method or use of any one of paragraphs 54-66, wherein the subject is human.

68. The method or use of any one of paragraphs 54-67, wherein the cancer is selected from the group consisting of breast cancer, bladder cancer, melanoma, prostate cancer, mesothelioma, lung cancer, testicular cancer, thyroid cancer, squamous cell carcinoma, glioblastoma, neuroblastoma, uterine cancer, colorectal cancer, and pancreatic cancer.

69. An isolated polynucleotide that comprises a nucleotide sequence that encodes at least one amino acid sequence selected from the group consisting of an antibody heavy chain variable region (V_{H}) and an antibody light chain variable region (V_{L}), wherein the V_{H} and the V_{L} comprise complementarity determine regions (CDR) identical to monoclonal antibody F90-10C5 CDRs.

70. A vector that comprises a polynucleotide according to paragraph 69.

71. A cell comprising a polynucleotide according to paragraph 69 or a vector according to paragraph 70, wherein (a) the cell expresses an antibody or antibody fragment containing the V_{H} and the V_{L}, and (b) the antibody or antibody fragment binds FGFR4.

72. A method of selecting an antibody or antibody fragment, wherein the method comprises:

(a) obtaining one or more antibodies or antibody fragments that bind FGFR4;

(b) screening the antibodies or antibody fragments in a tumor cell invasiveness assay;

and

(c) selecting an antibody that inhibits invasiveness in the assay by at least 50%.

73. The method of paragraph 72, wherein (b) comprises detecting invasion of tumor cells expressing FGFR4 in a three-dimensional collagen invasion assay using fibroblast growth factor-2 as a chemoattractant.

74. An isolated antibody or antibody fragment selected by the method of paragraph 72 or paragraph 73.

75. A hybridoma cell line deposited under Deutsche Sammlung von Mikroorganismen and Zellkulturen GmbH (DSMZ) Deposit Accession Number DSM ACC2967.

76. A hybridoma cell line deposited under DSMZ Deposit Accession Number DSM ACC2966.

77. A hybridoma cell line deposited under DSMZ Deposit Accession Number DSM ACC2965.

78. An isolated cell capable of producing antibody mAb F90-3B6.

79. The isolated cell of paragraph 78, wherein the cell is hybridoma F90-3B6 (DSMZ Deposit Accession Number DSM ACC2965).

80. An isolated cell capable of producing antibody mAb F90-10C5.

81. The isolated cell of paragraph 80, wherein the cell is hybridoma F90-10C5 (DSMZ Deposit Accession Number DSM ACC2967).

82. An isolated cell capable of producing antibody mAb F85-6C5.

83. The isolated cell of paragraph 82, wherein the cell is hybridoma F85-6C5 (DSMZ Deposit Accession Number DSM ACC2966).

84. An isolated antigenic peptide consisting of 5-25 amino acids of the amino acid sequence encoding FGFR4, wherein the peptide comprises the amino acid sequence set forth in any one of SEQ ID NOs: 5-9 or a fragment thereof.

85. An isolated polynucleotide encoding the antigenic peptide of paragraph 84.

86. A vector comprising the polynucleotide of paragraph 85.

87. An isolated cell comprising the vector of paragraph 86.

88. A composition comprising the peptide of paragraph 84 and an adjuvant.

89. The method of any one of paragraphs 56-63, comprising a step of determining the presence or absence of an FGFR4 allele that encodes FGFR4 R388 in the cancer, wherein the treatment is administered if the cancer has at least one FGFR4 allele that encodes FGFR4 R388.

90. A method of treating a mammalian subject comprising: selecting for treatment a mammalian subject diagnosed with or treated for cancer, wherein the cancer includes cells that contain at least one FGFR4 allele that encodes FGFR4 R388; and administering to the subject the composition of any one of claims 36-45 and 50, in an amount effective to modulate cancer cell invasion, ingrowth, or metastasis.

91. The method of paragraphs 89 or 90, wherein the presence or absence of an FGFR4 R388 allele is determined by assaying FGFR4 protein with an antibody or antibody fragment that differentially binds FGFR4 R388 and G388 alleles.

92. The method of paragraphs 89 or 90, wherein the presence or absence of an FGFR4 R388 allele is determined by assaying nucleic acid from the subject or from the cancer.

93. A method of treating a mammalian subject comprising: selecting for treatment a mammalian subject diagnosed with or treated for cancer; and administering to the subject a first anti-FGFR4 antibody or FGFR4-binding fragment thereof and a second anti-FGFR4 antibodies or FGFR4-binding fragment thereof, wherein the first anti-FGFR4 antibody or fragment inhibits FGF2-induced phosphorylation of FGFR4 R388, and wherein the second anti-FGFR4 antibody or fragment inhibits ligand-independent FGFR4 phosphorylation.

94. The method of paragraph 93, wherein the first and second antibodies or fragments thereof are separately administered, either simultaneously or sequentially, as a first composition containing the first antibody or fragment and a second composition containing the second antibody or fragment.

95. The method according to any one of paragraphs 90-94, further comprising administering to the subject a standard of care chemotherapy for the cancer.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. Where protein (e.g., antibody) therapy is described, embodiments involving polynucleotide therapy (using polynucleotides/vectors that encode the protein) are specifically contemplated, and the reverse also is true. Where embodiments of the invention are described with respect to a specific antibody, such as an FGFR4 monoclonal antibody, it should be appreciated that analogous embodiments involving antibody fragments, variants, and the like are specifically contemplated.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. With respect to aspects of the invention described or claimed with “a” or “an,” it should be understood that these terms mean “one or more” unless context unambiguously requires a more restricted meaning With respect to elements described as one or more within a set, it should be understood that all combinations within the set are contemplated.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### DESCRIPTION OF THE FIGURES

FIG. 1 is a graph illustrating the relative level of MMP2 activation (active/latent) (Y-axis) for various kinases (listed on X-axis) demonstrating greater than 2-fold induced MMP2 activation relative to mock transfected control cells.

FIG. 2 is a graph comparing absorbance (Y-axis) caused by ligand-receptor binding and concentration (nM) of potential binding blockers (X-axis).

FIG. 3A-C are illustrations of alternative views of the three dimensional structure of dimerized FGFR4, depicting the location of the epitope region bound by antibody F90-10C5 (SEQ ID NOs: 5-9) as beaded regions.

FIGS. 4A-4C are graphs comparing response units (Y-axis) and concentration (nM) of mAb 10C5 (also referred to herein as Ab F90-10C5) (FIG. 4A), mAb 6C5 (also referred to herein as Ab F85-6C5) (FIG. 4B), and mAb 3B6 (also referred to herein as Ab F90-3B6) (FIG. 4C) (X-axis).

FIG. 5 is a graph summarizing the number of collagen invasion foci (Y-axis) with treatment by control antibody, mAb 10C5, mAb 6C5, and mAb 3B6 (X-axis).

FIG. 6 depicts an immunoblot prepared from MDA-MB-231 cells transfected with expression vectors encoding VS-tagged FGFR4 R388 (FGFR4 R). The cells were pretreated with mAb F90-3B6, mAb F90-10C5, or a combination of mAb F90-3B6 and mAb F90-10C5 overnight, and left unstimulated (−) or incubated with FGF2 (+). Cell extracts were immunoprecipitated with antibodies against FGFR4 (IP:FGFR4) and immunoblotted using antibodies against the V5 tag (IB: V5) or phosphotyrosine residues (IB: pY).

FIG. 7 depicts an immunoblot prepared from COS-1 cells transfected with expression vectors encoding VS-tagged FGFR4 G388 (“FGFR4 G” or “FR4 G”), VS-tagged FGFR4 R388 (“FGFR4 R” or “FR4 R”), and FGFR1, alone or in combination. The cells were pretreated with mAb F85-6C5 or mAb F90-10C5, and incubated with FGF2 (+) or left unstimulated (−). Cell extracts were immunoprecipitated using anti-FGFR4 antibodies (IP:FGFR4) and immunoblotted using anti-phosphotyrosine antibodies (IB:pY), antibodies against FGFR1 (IB:FGFR1), or antibodies against the V5 tag (IB:VS). mAb F90-10C5 treatment inhibited FGF2-induced FGFR4 R388 phosphorylation and FGFR1 downregulation, whereas mAb F85-6C5 reduced ligand independent FGFR4 phosphorylation and FGFR4/FGFR1 heterodimerization after FGF2 stimulation.

### DETAILED DESCRIPTION OF THE INVENTION

The invention relates, at least in part, to the unexpected identification of certain anti-fibroblast growth factor receptor 4 (FGFR4) antibodies that inhibit invasion of cancer cells into surrounding tissue. While not wishing to be bound to a particular mechanism of action, the inventors surprisingly determined that FGFR4 is functionally linked with human membrane-type matrix metalloproteinase 1 (MT1-MMP), which is expressed in cancer and reactive cells. MT 1-MMP is largely sequestered in tissue microenvironments, enabling it to escape inactivation by many known MMP inhibitors. FGFR4 represents a novel target for MT1-MMP-mediated metastatic events. The invention provides an isolated antibody or fragment thereof that binds an FGFR4 and inhibits or downregulates cancer cell invasion, as well as methods of using the antibody or fragment thereof to modulate invasion, ingrowth, or metastasis of cancer cells. In this manner, antibodies of the invention have therapeutic utility to slow cancer progression.

### FGFR4

FGFR4 is one of four transmembrane receptor tyrosine kinases activated by FGF (Givol et al., FASEB J., 6: 3362-3369, 1992). The receptor is composed of three immunoglobulin (Ig)-like extracellular domains, a transmembrane domain, a tyrosine kinase, and a COOH-terminal tail (Givol et al., supra). At least two known human FGFR4 amino acid sequences have been reported, differing as a result of a mutation affecting codon 388, resulting in either a glycine (FGFR4 G388, SEQ ID NO: 1) or an arginine (FGFR4 R388, SEQ ID NO: 2) at this position. The allele with the R388 mutation correlates with aggressive tumor progression and is considered an indicator of poor clinical outcome (see, e.g., Bange et al., Cancer Res., 62(3): 840-7, 2002). The mutation results in substitution of a hydrophobic with a hydrophilic amino acid in the transmembrane domain of the protein. In this regard, a wild-type FGFR4 transmembrane domain comprises approximately the amino acid sequence RYTDIILYASGSLALAVLLLLAGLY (SEQ ID NO: 3), while the R388 mutant comprises a transmembrane domain comprising approximately the amino acid sequence RYTDIILYASGSLALAVLLLLARLY (SEQ ID NO: 4). The R388 FGFR4 mutant is further described in, e.g., Bange et al., supra; and U.S. Pat. No. 6,770,742, incorporated herein by reference.

### Antibodies and Fragments Thereof

Some embodiments or aspects of the invention relate to an antibody or fragment thereof that binds an extracellular epitope of FGFR4 (comprising the amino acid sequence of any FGFR4 polypeptide, including any naturally occurring isoforms or allelic variants of FGFR4) and inhibits cancer cell invasion. For example, the antibody or fragment thereof binds an FGFR4 expressed on the surface of a mammalian (e.g., human) cell. The antibody or fragment thereof can bind to an FGFR4 comprising the amino acid sequence set forth in SEQ ID NO: 1, which is commonly referred to as the FGFR4 G388 allele. Alternatively or in addition, the antibody or fragment thereof can bind an FGFR4 wherein the glycine at position 388 of wild-type FGFR4 is substituted with an arginine (the FGFR4 R388 allele) (SEQ ID NO: 2).

Preferably, the antibody or fragment thereof binds an extracellular epitope of a FGFR4. The three immunoglobulin (Ig)-like domains of FGFR4's extracellular region extend beyond the transmembrane domain into the extracellular space and, with reference to SEQ ID NOs: 1 and 2, comprise approximately amino acid residues 25-369 of the FGFR4 amino acid sequence. In certain aspects of the invention, the antibody or fragment thereof binds an extracellular epitope located in the region of the extracellular domain spanning amino acid residues 25-366, such as the first immunoglobulin-like domain of the extracellular region of FGFR4 (spanning approximately amino acid residues 50-107 of the FGFR4 amino acid sequence). The extracellular domain of FGFR4 is further described in Loo et al., Int. J. Biochem. Cell Biol., 32: 489-97, 2000; and Sorenson et al., J. Cell. Sci., 117: 1807-1819, 2004, the disclosures of which pertaining to FGFR4 are hereby incorporated by reference.

Any type of antibody is suitable in the context of the invention, including polyclonal, monoclonal, chimeric, humanized, or human versions having full length heavy and/or light chains. The invention also includes antibody fragments (and/or polypeptides that comprise antibody fragments) that retain FGFR4 binding characteristics of FGFR4 antibodies of the invention. Antibody fragments include antigen-binding regions and/or effector regions of the antibody, e.g., F(ab')₂, Fab, Fab', Fd, Fc, and Fv fragments (fragments consisting of the variable regions of the heavy and light chains that are non-covalently coupled), or single-domain antibodies (nanobodies). In general terms, a variable (V) region domain may be any suitable arrangement of immunoglobulin heavy (V_{H}) and/or light (V_{L}) chain variable domains. Thus, for example, the V region domain may be dimeric and contain V_{H}-V_{H}, V_{H}-V_{L}, or V_{L}-V_{L} dimers that bind FGFR4. If desired, the V_{H} and V_{L} chains may be covalently coupled either directly or through a linker to form a single chain Fv (scFv). For ease of reference, scFv proteins are referred to herein as included in the category “antibody fragments.” Similarly, antibody fragments may be incorporated into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9): 1126-1136, 2005) to inhibit cancer cell invasion. Additionally, the invention provides an isolated polypeptide that comprises a fragment of an antibody that binds an extracellular epitope of a fibroblast growth factor receptor-4 (FGFR4) that is expressed by mammalian cells. If desired, the antibody fragment is fused to a moiety with effector function (e.g., cytotoxic activity, immune recruitment activity, and the like), a moiety that facilitates isolation from a mixture (e.g., a tag), a detection label, or the like. It will be appreciated that the features of the inventive antibody or fragment thereof described herein extend also to a polypeptide comprising the antibody fragment.

The antibody or antibody fragment can be isolated from an immunized animal, synthetic, or genetically-engineered. Antibody fragments derived from an antibody can be obtained, e.g., by proteolytic hydrolysis of the antibody. For example, papain or pepsin digestion of whole antibodies yields a 5S fragment termed F(ab′)₂ or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1: 422 Academic Press (1967); Nisonoff et al., Arch. Biochem. Biophys., 89: 230-244, 1960; Porter, Biochem. J., 73: 119-127, 1959; U.S. Pat. No. 4,331,647; and by Andrews, S. M. and Titus, J. A. in Current Protocols in Immunology (Coligan et al., eds), John Wiley & Sons, New York (2003), pages 2.8.1-2.8.10 and 2.10A.1-2.10A.5.

An antibody or fragment thereof also can be genetically engineered such that the antibody or antibody fragment comprises, e.g., a variable region domain generated by recombinant DNA engineering techniques. For example, a specific antibody variable region can be modified by insertions, deletions, or changes in or to the amino acid sequences of the antibody to produce an antibody of interest. In this regard, polynucleotides encoding complementarity determining regions (CDRs) of interest are prepared, for example, by using polymerase chain reaction to synthesize variable regions using mRNA of antibody-producing cells as a template (see, for example, Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley-Liss, Inc. 1995); and Larrick et al., Methods: A Companion to Methods in Enzymology, 2: 106-110, 1991). Current antibody manipulation techniques allow construction of engineered variable region domains containing at least one CDR and, optionally, one or more framework amino acids from a first antibody and the remainder of the variable region domain from a second antibody. Such techniques are used, e.g., to humanize an antibody or to improve its affinity for a binding target.

“Humanized antibodies” are recombinant proteins in which complementary determining regions of monoclonal antibodies have been transferred from heavy and light variable chains of non-human immunoglobulin into a human variable domain. Constant regions need not be present, but if they are, they optionally are substantially identical to human immunoglobulin constant regions, i.e., at least about 85-90%, about 95% or more identical, in certain embodiments. Hence, in some instances, all parts of a humanized immunoglobulin, except possibly the CDR's, are substantially identical to corresponding parts of natural human immunoglobulin sequences. For example, in one aspect, humanized antibodies are human immunoglobulins (host antibody) in which hypervariable region residues of the host antibody are replaced by hypervariable region residues from a non-human species (donor antibody) such as mouse, rat, rabbit, or a non-human primate having the desired specificity, affinity, and capacity. Humanized antibodies such as those described herein can be produced using techniques known to those skilled in the art (Zhang et al., Molecular Immunology, 42(12): 1445-1451, 2005; Hwang et al., Methods, 36(1): 35-42, 2005; Dall'Acqua et al., Methods, 36(1): 43-60, 2005; Clark, Immunology Today, 21(8): 397-402, 2000, and U.S. Pat. Nos. 6,180,370; 6,054,927; 5,869,619; 5,861,155; 5,712,120; and 4,816,567, all of which are all hereby expressly incorporated herein by reference).

In one embodiment, the antibody is a human antibody, such as, but not limited to, an antibody having variable regions in which both the framework and CDR regions are derived from human germline immunoglobulin sequences as described, for example, in Kabat et al. (1991) Sequences of proteins of Immunological Interest, Fifth Edition, U.S. Department of Health and Human Services, NIH Publication No. 91-3242. If the antibody contains a constant region, the constant region also preferably is derived from human germline immunoglobulin sequences. Human antibodies may comprise amino acid residues not encoded by human germline immunoglobulin sequences to, e.g., enhance the activity of the antibody, but do not comprise CDRs derived from other species (i.e., a mouse CDR placed within a human variable framework region).

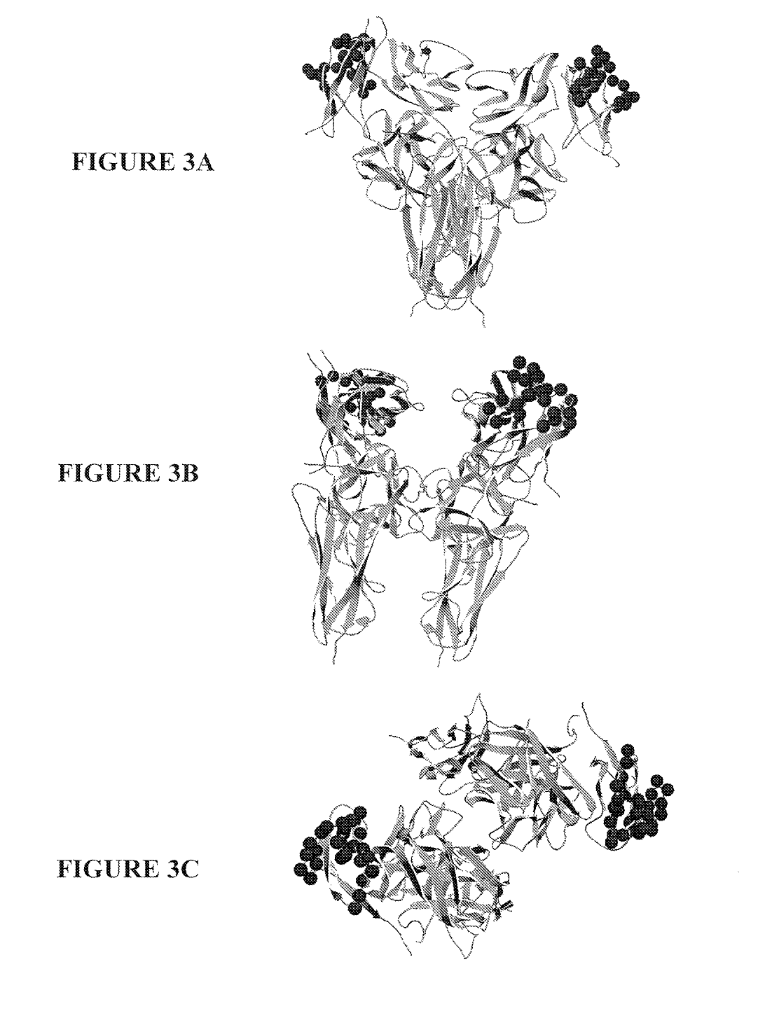

The antibody or fragment thereof binds any region of FGFR4 so long as cancer cell invasion is inhibited (or reduced) and/or one or more of the other desired activity parameters is retained. In one embodiment, the invention further provides an isolated antibody or antibody fragment that binds an epitope of FGFR4 that is bound by monoclonal antibody (mAb) F90-10C5 (also referred to herein as “10C5”), further described in the Examples below. mAb F90-10C5's binding activity is localized to the first immunoglobulin-like domain of the extracellular region of FGFR4 (see FIGS. 3A-3C). Surprisingly, mAb F90-10C5 recognizes a linear epitope within approximately amino acids 67-93 of the FGFR4 amino acid sequence, as determined by an immunoblotting array composed of a series of 15 amino acid fragments (the sequences of which overlapped by three amino acids) that spanned amino acids 67-93 of FGFR4's extracellular domain (excluding signal sequence). mAb F90-10C5 binds the following FGFR4 fragments: YKEGSRLAPAGRVRG (SEQ ID NO: 5); GSRLAPAGRVRGWRG (SEQ ID NO: 6); LAPAGRVRGWRGRLE (SEQ ID NO: 7); AGRVRGWRGRLEIAS (SEQ ID NO: 8); and VRGWRGRLEIASFLP (SEQ ID NO: 9). The isolated antibody or fragment thereof preferably binds a peptide comprising any one or more of the amino acid sequences set forth in SEQ ID NOs: 5-9. More preferably, the isolated antibody or fragment thereof binds a peptide comprising (or consisting of) the amino acid sequence of SEQ ID NO: 7.

Other antibodies that bind FGFR4 and inhibit cancer cell invasion also are suitable in the context of the invention. For example, in various embodiments, the invention includes administering an antibody or fragment thereof that (i) competes for binding with mAb F90-10C5, (ii) binds the region of FGFR4 recognized by mAb F90-10C5, or (iii) binds at or near amino acid residues 67-93 (e.g., amino acids residues 73-87 or amino acid residues 79-81) of the FGFR4 extracellular region, while inhibiting cancer cell invasion. If desired, the antibody fragment comprises all or part of the antigen-binding elements of an antibody, such as mAb F90-10C5, including the variable region of mAb F90-10C5 (or any other antibody of the invention). The antibody fragment can comprise all or part of the antigen-binding elements of an antibody while lacking all or part of the framework regions of an antibody. In this regard, the isolated antibody or fragment thereof comprises one, two, three, four, five, or six (i.e., all) complementary determining regions (CDRs) of an FGFR4-binding antibody that inhibits cancer cell invasion, e.g., mAb F90-10C5. Methods of identifying complementarity determining regions and specificity determining regions are known in the art and further described in, for example, Tamura et al., J. Immunol., 164: 1432-1441, 2000. In one embodiment, the antibody or fragment thereof is mAb F90-10C5 or an FGFR4-binding fragment thereof.

In the context of the invention, antibody binding refers to immuno-reacting between the variable regions of the antibody and an antigen as distinct from other protein-protein interactions (such as Staphylococcus aureus protein A interactions with immunoglobulins, for example). The antibody or fragment thereof preferably preferentially binds to FGFR4, meaning that the antibody or fragment thereof binds FGFR4 with greater affinity than it binds to an unrelated control protein. More preferably, the antibody or fragment thereof specifically recognizes and binds FGFR4 (or a portion thereof). “Specific binding” means that there is essentially no cross-reactivity with an unrelated control protein. In some variations of the invention, the antibody binds FGFR4 substantially exclusively (i.e., is able to distinguish FGFR4 from other known polypeptides (e.g., other FGFRs) by virtue of measurable differences in binding affinity). Depending on the embodiment, the antibody or fragment thereof binds to FGFR4 with an affinity that is at least 5, 10, 15, 20, 25, 50, 100, 250, 500, 1000, or 10,000 times greater than the affinity for an unrelated control protein. In other variations the antibody cross-reacts with other FGFR sequences. Screening assays to determine binding specificity/affinity of an antibody, as well as identify antibodies that compete for binding sites (i.e., cross-block binding of, e.g., mAb F90-10C5, to FGFR4), are well known and routinely practiced in the art. For example, binding affinity or cross-blocking can be determined using the methods described in the Examples. Competition binding assays employing a Biacore machine, which measures the extent of interactions using surface plasmon resonance technology, also are appropriate. Another suitable assay uses an ELISA-based approach to measure competition between antibodies in terms of their binding to FGFR4. For a comprehensive discussion of binding assays, see Harlow et al. (Eds), Antibodies A Laboratory Manual; Cold Spring Harbor Laboratory; Cold Spring Harbor, N.Y. (1988), Chapter 6. In general, an antibody that “competes” with or “cross-blocks” mAb F90-10C5 prevents the binding of mAb F90-10C5 to FGFR4 by 50% to 100% (e.g., 60%, 65%, 70%, 75%, 80%, 85%, 90%, or 95%).

### Materials and Methods for Producing Antibodies and Fragments Thereof

Antibodies according to the invention can be obtained by any suitable method, such as by immunization and cell fusion procedures as described herein and known in the art and/or screening libraries of antibodies or antibody fragments using FGFR4 extracellular domain epitopes described herein. Monoclonal antibodies of the invention are generated using a variety of known techniques (see, for example, Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). In one embodiment, the invention provides an isolated cell capable of producing antibody mAb F90-3B6, mAb F90-10C5, or mAb F85-6C5. Typically, monoclonal antibodies are produced by a hybridoma, and the invention provides a hybridoma that produces the inventive monoclonal antibody or antibody fragment. Hybridoma cell lines that produce antibodies F90-10C5, F85-6C5, and F90-3B6 are provided by the invention and have been deposited with the Deutsche Sammlung von Mikroorganismen and Zellkulturen GmbH (DSMZ), Mascheroder Wep lb. D-38124, Germany, under the provisions of the Budapest Treaty for the International Recognition of the Deposit of Microorganisms for the Purpose of Patent Procedure (“Budapest Treaty”), and assigned Deposit Accession Nos. DSM ACC2967, DSM ACC2966, and DSM ACC2965, respectively.

Likewise, human antibodies are generated by any of a number of techniques including, but not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. Methods for obtaining human antibodies from transgenic animals are further described, for example, in Bruggemann et al., Curr. Opin. Biotechnol., 8: 455-58, 1997; Jakobovits et al., Ann. N. Y Acad. Sci., 764: 525-35, 1995; Green et al., Nature Genet., 7: 13-21, 1994; Lonberg et al., Nature, 368: 856-859, 1994; Taylor et al., Int. Immun. 6: 579-591, 1994; and U.S. Pat. No. 5,877,397.

For example, human antibodies are obtained from transgenic animals that have been engineered to produce specific human antibodies in response to antigenic challenge. For example, International Patent Publication No. WO 98/24893 discloses transgenic animals having a human Ig locus, wherein the animals do not produce functional endogenous immunoglobulins due to the inactivation of endogenous heavy and light chain loci. Transgenic non-primate mammalian hosts capable of mounting an immune response to an immunogen, wherein the antibodies have primate constant and/or variable regions, and wherein the endogenous immunoglobulin encoding loci are substituted or inactivated, also have been described. International Patent Publication No. WO 96/30498 discloses the use of the Cre/Lox system to modify the immunoglobulin locus in a mammal, such as to replace all or a portion of the constant or variable region to form a modified antibody molecule. International Patent Publication No. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. Using a transgenic animal, such as a transgenic animal described herein, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in International Patent Publication No. WO 96/33735. The monoclonal antibodies can be tested for the ability to inhibit or neutralize the biological activity or physiological effect of the corresponding protein.

The invention provides materials for generating the inventive anti-FGFR4 antibodies and fragments thereof. For example, the invention provides an isolated cell (e.g., a hybridoma) that produces the inventive antibody or antibody fragment, such as hybridoma cell lines F90-10C5, F85-6C5, and F90-3B6 further described herein. The invention further relates to an isolated polynucleotide encoding the inventive antibody or antibody fragment. In one aspect of the invention, the isolated polynucleotide comprises a nucleotide sequence that encodes an antibody heavy chain variable region (V_{H}) and/or an antibody light chain variable region (V_{L}), wherein the V_{H} and the V_{L} comprise complementarity determining regions (CDRs) identical to monoclonal antibody F90-10C5 CDRs.

In a related embodiment, the invention provides a vector (e.g., an expression vector) comprising a polynucleotide of the invention to direct expression of the polynucleotide in a suitable host cell. Such vectors are useful, e.g., for amplifying the polynucleotides in host cells to create useful quantities thereof, and for expressing peptides, such as antibodies or antibody fragments, using recombinant techniques. In preferred embodiments, the vector is an expression vector wherein the polynucleotide of the invention is operatively linked to a polynucleotide comprising an expression control sequence. Autonomously replicating recombinant expression constructs such as plasmid and viral DNA vectors incorporating polynucleotides of the invention are specifically contemplated. Expression control DNA sequences include promoters, enhancers, and operators, and are generally selected based on the expression systems in which the expression construct is to be utilized. Preferred promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and preferably promote, homologous recombination in a host cell. Preferred expression constructs of the invention also include sequences necessary for replication in a host cell.

Exemplary expression control sequences include promoter/enhancer sequences, e.g., cytomegalovirus promoter/enhancer (Lehner et al., J. Clin. Microbiol., 29: 2494-2502, 1991; Boshart et al., Cell, 41: 521-530, 1985); Rous sarcoma virus promoter (Davis et al., Hum. Gene Ther., 4: 151, 1993); Tie promoter (Korhonen et al., Blood, 86(5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132: 2477-2478, 2007), for expression in the target mammalian cells, the promoter being operatively linked upstream (i.e., 5′) of the polypeptide coding sequence (the disclosures of the cited references is incorporated herein by reference in their entirety and particularly with respect to the discussion of expression control sequences). In another variation, the promoter is an epithelial-specific promoter or endothelial-specific promoter. The polynucleotides of the invention may also optionally include a suitable polyadenylation sequence (e.g., the SV40 or human growth hormone gene polyadenylation sequence) operably linked downstream (i.e., 3′) of the polypeptide coding sequence.

If desired, the polynucleotide of the invention also optionally comprises a nucleotide sequence encoding a secretory signal peptide fused in frame with the polypeptide sequence. The secretory signal peptide directs secretion of the polypeptide of the invention by the cells that express the polynucleotide, and is cleaved by the cell from the secreted polypeptide. The polynucleotide may further optionally comprise sequences whose only intended function is to facilitate large scale production of the vector, e.g., in bacteria, such as a bacterial origin of replication and a sequence encoding a selectable marker. However, if the vector is administered to an animal, such extraneous sequences are preferably at least partially cleaved. One can manufacture and administer polynucleotides for gene therapy using procedures that have been described in the literature for other transgenes. See, e.g., Isner et al., Circulation, 91: 2687-2692, 1995; and Isner et al., Human Gene Therapy, 7: 989-1011, 1996; incorporated herein by reference.

In some embodiments, polynucleotides of the invention further comprise additional sequences to facilitate uptake by host cells and expression of the antibody or fragment thereof (and/or any other peptide). In one embodiment, a “naked” transgene encoding an antibody or fragment thereof described herein (i.e., a transgene without a viral, liposomal, or other vector to facilitate transfection) is employed.

Vectors also are useful for “gene therapy” treatment regimens, wherein, for example, a polynucleotide encoding an antibody or fragment thereof is introduced into a subject suffering from or at risk of suffering from invasive cancers in a form that causes cells in the subject to express the antibody or fragment thereof in vivo. Any suitable vector may be used to introduce a polynucleotide that encodes an antibody or fragment thereof into the host. Exemplary vectors that have been described in the literature include replication deficient retroviral vectors, including but not limited to lentivirus vectors (Kim et al., J. Virol., 72(1): 811-816, 1998; Kingsman & Johnson, Scrip Magazine, October, 1998, pp. 43-46); parvoviral vectors, such as adeno-associated viral (AAV) vectors (U.S. Pat. Nos. 5,474,9351; 5,139,941; 5,622,856; 5,658,776; 5,773,289; 5,789,390; 5,834,441; 5,863,541; 5,851,521; 5,252,479; Gnatenko et al., J. Invest. Med., 45: 87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89: 2581-2584, 1992; Stratford Perricaudet et al., J. Clin. Invest., 90: 626-630, 1992; and Rosenfeld et al., Cell, 68: 143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. All of the foregoing documents are incorporated herein by reference in their entirety and particularly with respect to their discussion of expression vectors. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, the viral vector is rendered replication-deficient by, e.g., deleting or disrupting select genes required for viral replication.

Other non-viral delivery mechanisms contemplated include calcium phosphate precipitation (Graham and Van Der Eb, Virology, 52: 456-467, 1973; Chen and Okayama, Mol. Cell Biol., 7: 2745-2752, 1987; Rippe et al., Mol. Cell Biol., 10: 689-695, 1990) DEAE-dextran (Gopal, Mol. Cell Biol., 5: 1188-1190, 1985), electroporation (Tur-Kaspa et al., Mol. Cell Biol., 6: 716-718, 1986; Potter et al., Proc. Nat. Acad. Sci. USA, 81: 7161-7165, 1984), direct microinjection (Harland and Weintraub, J. Cell Biol., 101: 1094-1099, 1985, DNA-loaded liposomes (Nicolau and Sene, Biochim. Biophys. Acta, 721: 185-190, 1982; Fraley et al., Proc. Natl. Acad. Sci. USA, 76: 3348-3352, 1979; Felgner, Sci Am., 276(6): 102-6, 1997; Felgner, Hum Gene Ther., 7(15): 1791-3, 1996), cell sonication (Fechheimer et al., Proc. Natl. Acad. Sci. USA, 84: 8463-8467, 1987), gene bombardment using high velocity microprojectiles (Yang et al., Proc. Natl. Acad. Sci USA, 87: 9568-9572, 1990), and receptor-mediated transfection (Wu and Wu, J. Biol. Chem., 262: 4429-4432, 1987; Wu and Wu, Biochemistry, 27: 887-892, 1988; Wu and Wu, Adv. Drug Delivery Rev., 12: 159-167, 1993).

The expression vector (or the antibody or fragment thereof discussed herein) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver diseases, targeted diagnosis and therapy using specific receptors and ligands, Wu G, Wu C ed., New York: Marcel Dekker, pp. 87-104 (1991)). The addition of DNA to cationic liposomes causes a topological transition from liposomes to optically birefringent liquid-crystalline condensed globules (Radler et al., Science, 275(5301): 810-814, 1997). These DNA-lipid complexes are potential non-viral vectors for use in gene therapy and delivery.

Liposome-mediated nucleic acid delivery and expression of foreign DNA in vitro has been successful. Also contemplated in the invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda et al., Science, 243: 375-378, 1989). In other embodiments, the liposome is complexed or employed in conjunction with nuclear nonhistone chromosomal proteins (HMG-1) (Kato et al., J. Biol. Chem., 266: 3361-3364, 1991). In yet further embodiments, the liposome are complexed or employed in conjunction with both HVJ and HMG-1. Such expression constructs have been successfully employed in transfer and expression of nucleic acid in vitro and in vivo. In some variations of the invention, an FGFR4 targeting moiety, such as an FGFR4 antibody or fragment, is included in the liposome to target the liposome to cells (such as cancer cells) expressing FGFR4 on their surface.

Transferring a naked DNA expression construct into cells can be accomplished using particle bombardment, which depends on the ability to accelerate DNA coated microprojectiles to a high velocity allowing them to pierce cell membranes and enter cells without killing them (Klein et al., Nature, 327: 70-73, 1987). Several devices for accelerating small particles have been developed. One such device relies on a high voltage discharge to generate an electrical current, which in turn provides the motive force (Yang et al., Proc. Natl. Acad. Sci USA, 87: 9568-9572, 1990). The microprojectiles used have consisted of biologically inert substances such as tungsten or gold beads.

In embodiments employing a viral vector, preferred polynucleotides still include a suitable promoter and polyadenylation sequence as described above. Moreover, it will be readily apparent that, in these embodiments, the polynucleotide further includes vector polynucleotide sequences (e.g., adenoviral polynucleotide sequences) operably connected to the sequence encoding a polypeptide of the invention.

The invention further provides a cell that comprises the polynucleotide or the vector, e.g., the cell is transformed or transfected with a polynucleotide encoding the inventive antibody or fragment thereof or a vector comprising the polynucleotide. In certain aspects of the invention, the cell expresses an anti-FGFR4 antibody or antibody fragment containing the V_{H} and the V_{L} comprising CDRs identical to those of mAb F90-10C5. The cell may be a prokaryotic cell, such as Escherichia coli (see, e.g., Pluckthun et al., Methods Enzymol., 178: 497-515, 1989), or a eukaryotic host cell, such as an animal cell (e.g., a myeloma cell, Chinese Hamster Ovary cell, or hybridoma cell), yeast (e.g., Saccharomyces cerevisiae), or a plant cell (e.g., a tobacco, corn, soybean, or rice cell). Use of mammalian host cells is expected to provide for such translational modifications (e.g., glycosylation, truncation, lipidation, and phosphorylation) that may be desirable to confer optimal biological activity on recombinant expression products. Similarly, the invention embraces polypeptides that are glycosylated or non-glycosylated and/or have been covalently modified to include one or more water soluble polymer attachments such as polyethylene glycol, polyoxyethylene glycol, or polypropylene glycol.

Polynucleotides of the invention may be introduced into the host cell as part of a circular plasmid, or as linear DNA comprising an isolated protein coding region or a viral vector. Methods for introducing DNA into the host cell, which are well known and routinely practiced in the art, include transformation, transfection, electroporation, nuclear injection, or fusion with carriers such as liposomes, micelles, ghost cells, and protoplasts. As stated above, such host cells are useful for amplifying the polynucleotides and also for expressing the polypeptides of the invention encoded by the polynucleotide. The host cell may be isolated and/or purified. The host cell also may be a cell transformed in vivo to cause transient or permanent expression of the polypeptide in vivo. The host cell may also be an isolated cell transformed ex vivo and introduced post-transformation, e.g., to produce the polypeptide in vivo for therapeutic purposes. The definition of host cell explicitly excludes a transgenic human being.

Particular methods for producing antibodies from polynucleotides are generally well known and routinely used. For example, basic molecular biology procedures are described by Maniatis et al., Molecular Cloning, A Laboratory Manual, 2nd ed., Cold Spring Harbor Laboratory, New York, 1989 (see also Maniatis et al, 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, UK, 1992); and Current Protocols in Molecular Biology, Ausubel ed., Wiley Interscience, New York, 1999).

The invention further provides a peptide comprising (or consisting of) amino acids 67-93 of FGFR4 or a subregion of amino acids 67-93 of FGFR4. In this regard, the invention provides a peptide comprising (or consisting of) the amino acid sequence YKEGSRLAPAGRVRG (SEQ ID NO: 5); GSRLAPAGRVRGWRG (SEQ ID NO: 6); LAPAGRVRGWRGRLE (SEQ ID NO: 7); AGRVRGWRGRLEIAS (SEQ ID NO: 8); or VRGWRGRLEIASFLP (SEQ ID NO: 9). The peptides of the invention may be used to, for example, generate antibodies against FGFR4 and/or identify antibodies for anti-FGFR4 activity. In addition, the invention contemplates use of the peptides as immunogens for, e.g., stimulating the immune system against tumor cells displaying FGFR4. In one aspect, the invention provides an isolated antigenic peptide consisting of 5-25 amino acids of an amino acid sequence encoding FGFR4, wherein the peptide comprises the amino acid sequence set forth in any one of SEQ ID NOs: 5-9 or a fragment thereof. The invention also provides a composition comprising any of the foregoing peptides and one or more excipient(s), adjuvant(s), chemotherapeutic agent(s), and the like. It will be appreciated the materials and methods for generating an antibody or fragment thereof also apply to the peptides described herein. For example, the invention provides a polynucleotide that encodes any of the foregoing peptides, a vector comprising the polynucleotide, and an isolated cell comprising the polynucleotide (optionally incorporated into a vector).

It will be understood that the polynucleotide, vector, and cell of the invention can be used in methods of inhibiting cancer cell invasion in vitro and in vivo (e.g., in a method of treating cancer in a subject).

### Inhibiting Cancer Cell Invasion/Methods of Treatment

The antibody or antibody fragment binds FGFR4 and inhibits cancer cell invasion. As used herein, “cancer cell invasion” refers to ingrowth of cancer cells into surrounding tissue or collagen or fibrin-rich interstitial and temporary matrixes. In this regard, the invention also provides a method of modulating invasion, ingrowth, or metastasis of cancer cells, wherein the method comprises contacting a population of cancer cells with the inventive antibody or fragment thereof (e.g., a composition comprising the antibody or fragment thereof) in an amount effective to modulate cancer cell invasion, ingrowth, or metastasis. When the cancer cells are present in a mammalian subject, the population of cancer cells is contacted by administering the composition comprising the inventive antibody or fragment thereof to the mammalian subject. In vivo, cancer cell invasion and metastasis is a multifaceted process requiring degradation of extracellular matrix, including basement membrane and collagen-rich interstitial matrixes, as well as active cell migration from a primary tumor. The antibody or fragment thereof blocks one or more of the processes associated with cancer cell invasion in vivo, such as extracellular matrix degradation. Preferably, the antibody or fragment thereof inhibits (i.e., downregulates or slows) both extracellular matrix (collagen and basement membrane) degradation and cell migration in a three-dimensional tissue environment.

The efficacy of the antibody to inhibit cancer cell invasion is demonstrated using an in vitro invasiveness assay, and preferably confirmed in an animal model for cancer. In this regard, the invention provides a method of identifying an antibody or antibody fragment, wherein the method comprises (a) obtaining one or more antibodies or antibody fragments that bind FGFR4; (b) screening the antibodies or antibody fragments in a tumor cell invasiveness assay; and (c) identifying an antibody that inhibits invasiveness in the assay by, e.g., at least 50%. An exemplary in vitro dual-chamber tumor cell invasiveness assay is described in the Examples. In some variations, the cells used in the invasiveness assay co-express the FGFR4 with other receptors, such as FGFR1. In some variations, the receptor(s) of interest are recombinantly expressed. In other variations, the cells are isolated from a tumor (primary isolates) or are from a tumor cell line that expresses the receptor(s) of interest.

In an exemplary assay, cancer cells (e.g., MDA-MB-231 cells) expressing FGFR4 (e.g., the FGFR4 R388 protein) are applied to a three-dimensional culture (e.g., collagen) gel. Optionally, a chemoattractant, such as FGF2, is applied (to the bottom chamber if a dual-chamber format is employed) to activate cell migration. Invasion can be determined by measuring the number of cells migrated into the culture gel, or by measuring the length of the cellular arms reaching into the gel. A decrease in cell invasion into the culture gel mediated by a candidate antibody, compared to invasion in the absence of the antibody, is indicative of an antibody or fragment thereof that inhibits cancer cell invasion. Tumor cell invasion assays are further described in, e.g., Puiffe et al., Neoplasia, 9(10): 820-829, 2007; Alonso-Escolano et al., J. Pharm. Exper. Ther., 318: 373-380, 2006; and Keese et al., BioTechniques, 33: 842-850, 2002. Antibodies or fragments thereof identified by the method are provided.

It will be appreciated that antibodies or fragments thereof can be characterized using other assays known in the art, such as those described in the Examples. For example, FGFR4 activation can be examined by detecting receptor phosphorylation using, e.g., immunoprecipitation and immunoblotting techniques. In certain aspects, the inventive antibody or fragment thereof inhibits or reduces ligand independent or ligand dependent (e.g., fibroblast growth factor 2 (FGF2)-induced) phosphorylation of FGFR4. Similar techniques can be employed to examine the effect of an antibody or fragment thereof on co-localization of FGFR4 and MT1-MMP in cells.

Additionally, the ability of an antibody or fragment thereof to inhibit cancer cell invasion in vivo can be determined using any suitable animal model, such as a bone invasion model (see, e.g., Kang, Cancer Cell, 3: 537-549, 2003; and Pauli et al., Cancer Research, 40: 4571-4580, 1980), an animal model of bladder carcinoma invasion (Mohammed et al., Mol. Cancer Ther., 2(2): 183-188, 2003; and Kameyama et al., Carcinogenesis., 14(8): 1531-1535, 1993), a mouse melanoma metastasis model (Lee et al., Cancer Chemother. Pharmacol., 57(6): 761-71, 2006), or a screening assay employing modified chorioallantoic membrane (see, e.g., Ossowski, J. Cell Biol., 107(6): 2437-2445, 1988). Methods of monitoring metastasis in a human patient are well known and include, for instance, examination of tissue biopsies to detect metastatic cells.

“Inhibiting,” “blocking,” or “impeding” cancer cell invasion does not require a 100% abolition of invasion or metastasis. Any decrease in cancer cell invasion constitutes a beneficial biological effect in a subject. In this regard, the antibody or fragment thereof can inhibit cell invasion (e.g., MDA-MB-231 cell invasion of a 3-D collagen tumor cell invasiveness assay) by, e.g., at least about 5% (at least about 10%, at least about 20%, or at least about 25%) compared to levels of cancer cell invasion observed in the absence of the antibody or fragment thereof (e.g., in a biologically-matched control subject, specimen, or cell culture that is not exposed to the antibody or fragment thereof). In some embodiments, cell invasion is reduced by at least about 50%, e.g., by at least about 60%, at least about 70%, at least about 80%, at least about 90%, or at least about 95%. In in vitro or animal models or in clinical trials involving repetition, inhibition of invasiveness can be confirmed using standard statistical analyses to confirm that results are statistically significant (e.g., to a level of significance of p<0.05).

In addition, the invention provides a method of treating cancer in a subject (e.g., a mammal, such as a human). The method comprises administering to the subject the inventive antibody or fragment thereof in an amount effective to treat cancer. “Treating cancer” encompasses inhibiting or arresting the development or progression of a disorder marked by abnormal cell or tissue growth or proliferation, particularly those with metastatic potential. “Treating cancer” also encompasses impeding the progress of invasive growth in surrounding tissue, thereby slowing cancer cell spread to distant organs and the growth of secondary tumors (metastases). “Treating cancer” also encompasses alleviating, in whole or in part, a disorder marked by hyperproliferation. Any cancer with metastatic potential or an invasive aspect and that expresses FGFR4 is suitable for treatment in the context of the invention. Indeed, in certain embodiments, the method comprises administering the inventive antibody or fragment thereof to a patent diagnosed with metastatic cancer. Exemplary cancers include cancers of the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid). In some embodiments, the cancer of the inventive method is breast cancer, bladder cancer, melanoma, prostate cancer, colorectal cancer, thyroid cancer, glioma, mesothelioma, lung cancer, testicular cancer, or pancreatic cancer. The R388 FGFR4 mutant has been detected in cell lines derived from breast tumors, squamous cell carcinoma, glioblastomas, neuroblastomas and uterine cancer (see U.S. Pat. No. 6,770,742); thus, the materials and methods of the invention are particularly suitable for the treatment of those disorders.

The progress of the inventive method in treating cancer (e.g., impeding cancer cell invasion in surrounding tissues) can be ascertained using any suitable method, such as those methods described herein and currently used in the clinic to track cancer progress. If desired, the efficacy of the inventive method is determined by detection of new tumors, detection of tumor antigens or markers, biopsy, positron emission tomography (PET) scans, survival, disease progression-free survival, time to disease progression, quality of life assessments such as the Clinical Benefit Response Assessment, and the like, all of which can point to the overall progression (or regression) of cancer in a human.

In some embodiments of the invention, the method of the invention comprises determining the presence or absence of an FGFR4 allele that encodes FGFR4 R388 in the cancer. Depending on the particular embodiment, the treatment is administered if the cancer has at least one FGFR4 allele that encodes FGFR4 R388. Thus, in one aspect, the invention provides a method of treating a mammalian subject, wherein the method comprises selecting for treatment a mammalian subject diagnosed with or treated for cancer, wherein the cancer includes cells that contain at least one FGFR4 allele that encodes FGFR4 R388; and administering to the subject a composition comprising the antibody or fragment thereof (or nucleic acid encoding the antibody or fragment thereof) in an amount effective to modulate cancer cell invasion, ingrowth, or metastasis.

The presence or absence of an FGFR4 R388 allele in a biological sample can be determined using a variety of techniques. Samples typically are isolated from blood, serum, urine, or tissue biopsies from, e.g., muscle, connective tissue, nerve tissue, and the like. Once obtained, cells from the sample are examined to detect the presence or absence of FGFR4 R388. One method for identifying FGFR4 R388 comprises assaying nucleic acid (e.g., obtaining nucleic acid sequence data) from a biological sample taken from a subject (e.g., a cancer specimen or tissue biopsy). Genomic DNA, RNA, or cDNA is obtained from a biological sample and, optionally, the nucleic acid encoding FGFR4 is amplified by polymerase chain reaction (PCR). The DNA, RNA, or cDNA sample is then examined. The presence of FGFR4 R388 can be determined by sequence-specific hybridization of a nucleic acid probe specific for the R388 allele. One of skill in the art has the requisite knowledge and skill to design a probe so that sequence-specific hybridization will occur only if the biological sample contains an FGFR4 R388 coding sequence. Alternatively or in addition, the presence or absence of FGFR4 R388 is determined by directly sequencing DNA or RNA obtained from a subject.

In one aspect, the presence or absence of an FGFR4 R388 allele in a biological sample (e.g., a cell) is determined by assaying FGFR4 protein with an antibody or antibody fragment that differentially binds FGFR4 R388 and G388 alleles. The term “differentially binds” refers to the antibody's ability to distinguish between the R388 and G388 FGFR4 proteins. For example, an antibody or fragment thereof that differentially binds FGFR4 R388 binds the protein with greater affinity (e.g., at least 10, 15, 20, 25, 50, or 100 times greater affinity) than it binds to FGFR4 G388. Exemplary methods for detecting FGFR4 R388 protein include, but are not limited to, immunoassays, e.g., immunofluorescent immunoassays, immunoprecipitations, radioimmunoasays, ELISA, Western blotting, and fluorescence activated cell sorting (FACS). These methods comprise contacting a biological sample with an antibody or fragment thereof that differentially binds FGFR4 R388, and detecting antibody binding to FGFR4 R388.

### Non-Antibody Based Inhibitors

Some variations of the invention include use of non-antibody-based inhibitors against FGFR4 and, optionally, MT1-MMP. Matrix metalloproteinases (MMP) constitute a family of enzymes responsible for extracellular matrix degradation in cancer tissue (Nagase et al., J. Biol. Chem., 274: 21491-21494, 1999; Nelson et al., J. Clin. Oncol., 18: 1135-1149, 2000). MMPs are zinc-dependent multidomain endopeptidases that, with few exceptions, share a basic structural organization comprising propeptide, catalytic, hinge, and C-terminal (hemopexin-like) domains (Nagase et al., supra; Massova et al., FASEB J., 12: 1075-1095, 1998). All MMPs are produced in a latent form (pro-MMP) requiring activation for catalytic activity, a process that is usually accomplished by proteolytic removal of the propeptide domain.

MT1-MMP (MMP-14) is a multifunctional enzyme that degrades a variety of extracellular matrix components including fibrillar collagen and fibrin (Pei et al., J. Biol. Chem., 271: 9135-9140, 1996; d'Ortho et al., FEBS Lett., 421: 159-164, 1998; Ohuchi et al., J. Biol. Chem., 272: 2446-2451, 1997). In addition, both MMP-2 and MT1-MMP are associated with metastatic potential in many human cancers, and enhance tumor cell invasion in experimental systems. MT-MMP1 is frequently upregulated in both cancer cells and reactive stromal cells in various forms of cancer, and cancer cells overexpressing MT1-MMP invade, proliferate, and metastasize in nude mice at remarkably higher rates than control cells. The amino acid sequence of MT1-MMP is provided in SEQ ID NO: 10.

FGFR4 or MT1-MMP inhibitors modulate activity by targeting FGFR4 or MT1-MMP directly, i.e., at the protein level, targeting transcription or translation of FGFR4 or MT1-MMP, or targeting a downstream molecule required for realization of FGFR4 or MT1-MMP function. On the nucleic acid level, inhibitors inactivate or disrupt FGFR4 or MT1-MMP coding sequence. Inhibition may also block transcription or translation by targeting genomic DNA or FGFR4 mRNA, FGFR4 ligand mRNA, MT1-MMP mRNA and/or mRNA of downstream targets. In this regard, antisense therapy is one method for inhibiting expression, described below with particular reference to FGFR4 and MT1-MMP with the understanding that the description is equally suitable for other gene targets.

Antisense oligonucleotides negatively regulate FGFR4 (or MT1-MMP) expression via hybridization to messenger RNA (mRNA) encoding FGFR4 (or MT1-MMP). The nucleic acid sequences encoding FGFR4 G388 protein and FGFR4 R388 protein are known, e.g., as reported in GenBank Accession No. X57205 for the nucleic acid sequence of FGFR4 G388 (SEQ ID NO: 11). The nucleic acid sequence of FGFR4 R388 is provided in SEQ ID NO: 12. Likewise, the nucleic acid sequence encoding MT1-MMP is known in the art, e.g., as reported in GenBank Accession No. X90925 (SEQ ID NO: 13). These sequences may be used to prepare and optimize antisense molecules using any methods known in the art. In this regard, the invention includes use of an inhibitor nucleic acid that hybridizes with MT1-MMP (or FGFR4) genomic DNA or mRNA and inhibits MT1-MMP (or FGFR4) transcription or translation. All classes of nucleic acid inhibitor described herein can be used alone or in combination with other inhibitor substances described herein (see section below relating to combination therapies).

In one aspect, antisense oligonucleotides at least 5 to about 50 nucleotides in length, including all lengths (measured in integer number of nucleotides) in between, which specifically hybridize to mRNA encoding FGFR4 or MT1-MMP and inhibit mRNA expression, and as a result FGFR4 or MT1-MMP protein expression, are contemplated for use in the inventive method. Antisense oligonucleotides include those comprising modified internucleotide linkages and/or those comprising modified nucleotides which are known in the art to improve stability of the oligonucleotide, i.e., make the oligonucleotide more resistant to nuclease degradation, particularly in vivo. It is understood in the art that, while antisense oligonucleotides that are perfectly complementary to a region in the target polynucleotide possess the highest degree of specific inhibition, antisense oligonucleotides which are not perfectly complementary, i.e., those which include a limited number of mismatches with respect to a region in the target polynucleotide, also retain high degrees of hybridization specificity and therefore inhibit expression of the target mRNA. Accordingly, the invention includes methods using antisense oligonucleotides that are perfectly complementary to a target region in a polynucleotide encoding FGFR4 or MT1-MMP, as well as methods that utilize antisense oligonucleotides that are not perfectly complementary, i.e., include mismatches, to a target region in the target polynucleotide to the extent that the mismatches do not preclude specific hybridization to the target region in the target polynucleotide. Preparation and use of antisense compounds is described in U.S. Pat. No. 6,277,981, the disclosure of which is incorporated herein by reference in its entirety.

Another class of therapeutics for inhibiting expression of the target genes described herein is ribozymes. Ribozyme inhibitors include a nucleotide region which specifically hybridizes to a target polynucleotide and an enzymatic moiety that digests the target polynucleotide. Specificity of ribozyme inhibition is related to the length the antisense region and the degree of complementarity of the antisense region to the target region in the target polynucleotide. The invention therefore includes use of ribozyme inhibitors of FGFR4 or MT1-MMP comprising antisense regions from 5 to about 50 nucleotides in length, including all nucleotide lengths in between, that are perfectly complementary, as well as antisense regions that include mismatches to the extent that the mismatches do not preclude specific hybridization to the target region in the target FGFR4- or MT1-MMP-encoding polynucleotides. Ribozymes useful in methods of the invention include those comprising modified internucleotide linkages and/or those comprising modified nucleotides which are known in the art to improve stability of the oligonucleotide, i.e., make the oligonucleotide more resistant to nuclease degradation, particularly in vivo, to the extent that the modifications do not alter the ability of the ribozyme to specifically hybridize to the target region or diminish enzymatic activity of the molecule. Because ribozymes are enzymatic, a single molecule is able to direct digestion of multiple target molecules thereby offering the advantage of being effective at lower concentrations than non-enzymatic antisense oligonucleotides. Preparation and use of ribozyme technology is described in U.S. Pat. Nos. 6,696,250; 6,410,224; and 5,225,347, the disclosures of which are incorporated herein by reference in their entireties.

Another class of therapeutics for inhibiting expression (and therefore activity) of target genes/pathways described herein is interfering RNA technology, also known as RNA interference (RNAi) or short interfering RNA (siRNA). Using the knowledge of the sequence of target genes such as FGFR4 or MT1-MMP, siRNA molecules are formed that interfere with the expression of the genes. siRNA describes a technique by which post-transcriptional gene silencing (PTGS) is induced by the direct introduction of double stranded RNA (dsRNA: a mixture of both sense and antisense strands) (Fire et al., Nature, 391: 806-811, 1998). Current models of PTGS indicate that short stretches of interfering dsRNAs (21-23 nucleotides; siRNA also known as “guide RNAs”) mediate PTGS. The siRNAs are apparently produced by cleavage of dsRNA introduced directly or via a transgene or virus. These siRNAs may be amplified by an RNA-dependent RNA polymerase (RdRP) and are incorporated into the RNA-induced silencing complex (RISC), guiding the complex to the homologous endogenous mRNA, where the complex cleaves the transcript. It is contemplated that RNAi may be used to disrupt the expression of a gene in a tissue-specific manner. By placing a gene fragment encoding the desired dsRNA behind an inducible or tissue-specific promoter, it should be possible to inactivate genes at a particular location within an organism or during a particular stage of development.

Also contemplated is double-stranded RNA (dsRNA) wherein one strand is complementary to a target region in a target FGFR4- or MT1-MMP-encoding polynucleotides. In general, dsRNA molecules of this type less than 30 nucleotides in length are referred to in the art as short interfering RNA (siRNA). The invention also includes, however, use of dsRNA molecules longer than 30 nucleotides in length, and in certain aspects of the invention, these longer dsRNA molecules can be about 30 nucleotides in length up to 200 nucleotides in length and longer, and including all length dsRNA molecules in between. As with other RNA inhibitors, complementarity of one strand in the dsRNA molecule can be a perfect match with the target region in the target polynucleotide, or may include mismatches to the extent that the mismatches do not preclude specific hybridization to the target region in the target FGFR4- or MT1-MMP-encoding polynucleotides. As with other RNA inhibition technologies, dsRNA molecules include those comprising modified internucleotide linkages and/or those comprising modified nucleotides which are known in the art to improve stability of the oligonucleotide, i.e., make the oligonucleotide more resistant to nuclease degradation, particularly in vivo. Exemplary lentiviral shRNA constructs targeting MT1-MMP include TRCN0000050854 (GenBank Accession No. NM 004995) and TRCN0000050585 (GenBank Accession No. NM 006703) from Open Biosystems (Huntsville, Alabama) (catalog nos. catalog RHS3979-9618053 and RHS3979-9617784; described further in Tatti et al., Exp. Cell. Res., 314(13): 2501-14, 2008). HP Validated siRNA SI03648841 (SEQ ID NO: 14) from Qiagen (Hilden, Germany) also effectively downregulates MT1-MMP. Exemplary FGFR4-targeting siRNAs include HP Validated siRNA SIO2659979 (SEQ ID NO: 15), HP Validated siRNA SIO2665306, and HP GenomeWide siRNA SI00031360 (SEQ ID NO: 16) from Qiagen (Hilden, Germany). Preparation and use of RNAi compounds is described in U.S. Patent Application No. 20040023390, the disclosure of which is incorporated herein by reference in its entirety.

The invention further contemplates methods wherein inhibition of FGFR4 or MT1-MMP is effected using RNA lasso technology. Circular RNA lasso inhibitors are highly structured molecules that are inherently more resistant to degradation and therefore do not, in general, include or require modified internucleotide linkage or modified nucleotides. The circular lasso structure includes a region that is capable of hybridizing to a target region in a target polynucleotide, the hybridizing region in the lasso being of a length typical for other RNA inhibiting technologies. As with other RNA inhibiting technologies, the hybridizing region in the lasso may be a perfect match with the target region in the target polynucleotide, or may include mismatches to the extent that the mismatches do not preclude specific hybridization to the target region in the target FGFR4- or MT1-MMP-encoding polynucleotides. Because RNA lassos are circular and form tight topological linkage with the target region, inhibitors of this type are generally not displaced by helicase action unlike typical antisense oligonucleotides, and therefore can be utilized as dosages lower than typical antisense oligonucleotides. Preparation and use of RNA lassos is described in U.S. Pat. No. 6,369,038, the disclosure of which is incorporated herein by reference in its entirety.

Anti-sense RNA and DNA molecules, ribozymes, RNAi and triple helix molecules directed against FGFR4 or MT1-MMP can be prepared by any method known in the art for the synthesis of DNA and RNA molecules. These include techniques for chemically synthesizing oligodeoxyribonucleotides well known in the art including, but not limited to, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably or transiently into cells.

Aptamers are another nucleic acid based method for interfering with the interaction of FGFR4 or MT1-MMP is the use of an aptamer. Aptamers are DNA or RNA molecules that have been selected from random pools based on their ability to bind other molecules. Aptamers have been selected which bind nucleic acid, proteins, small organic compounds, and even entire organisms. Methods and compositions for identifying and making aptamers are known to those of skill in the art and are described e.g., in U.S. Pat. No. 5,840,867 and U.S. Pat. No. 5,582,981 each incorporated herein by reference. Aptamers that bind FGFR4 or MT1-MMP are specifically contemplated to be useful in the present therapeutic embodiments.

Recent advances in the field of combinatorial sciences have identified short polymer sequences with high affinity and specificity to a given target. For example, SELEX technology has been used to identify DNA and RNA aptamers with binding properties that rival mammalian antibodies, the field of immunology has generated and isolated antibodies or antibody fragments which bind to a myriad of compounds and phage display has been utilized to discover new peptide sequences with very favorable binding properties. Based on the success of these molecular evolution techniques, it is certain that molecules can be created which bind to any target molecule. A loop structure is often involved with providing the desired binding attributes as in the case of: aptamers which often utilize hairpin loops created from short regions without complimentary base pairing, naturally derived antibodies that utilize combinatorial arrangement of looped hyper-variable regions, and new phage display libraries utilizing cyclic peptides that have shown improved results when compared to linear peptide phage display results. Thus, sufficient evidence has been generated to suggest that high affinity ligands can be created and identified by combinatorial molecular evolution techniques. For the present invention, molecular evolution techniques can be used to isolate binding constructs specific for ligands described herein. For more on aptamers, see generally, Gold et al., J. Biotechnol., 74: 5-13, 2000. Relevant techniques for generating aptamers may be found in U.S. Pat. No. 6,699,843, which is incorporated by reference in its entirety.

In some embodiments, the aptamer may be generated by preparing a library of nucleic acids; contacting the library of nucleic acids with a target, e.g., FGFR4 or MT1-MMP, wherein nucleic acids having greater binding affinity for the target (relative to other library nucleic acids) are selected and amplified to yield a mixture of nucleic acids enriched for nucleic acids with relatively higher affinity and specificity for binding to the target. The processes may be repeated, and the selected nucleic acids mutated and re-screened, whereby a target aptamer is identified.

Other inhibitors target FGFR4 or MT1-MMP directly, i.e., at the protein level. In this regard, chemical compound inhibitors of FGFR4 or MT1-MMP are contemplated. Small molecule compounds (i.e., compounds having a molecular weight of less than 1000 Daltons, typically between 300 and 700 Daltons) are generally preferred because the reduced size renders the molecule more accessible for uptake by a target cell. Synthetic inhibitors of FGFR4 include PD173074 (Pfizer; Ezzat et al, Clinical Cancer Res., 11: 1336-1341, 2005, and Kwabi-Addo et al., Endocrine-Related Cancer, 11; 709-724, 2004). Synthetic inhibitors capable of blocking MT1-MMP activity include Ro-28-2653, described in Maquoi et al., Clin. Cancer Res., 15: 4038-47 (2004). Synthetic inhibitors are further described in Nisato et al., Cancer Res., 65(20): 9377-9387, 2005; and Galvez et al., J. Biol. Chem., 276: 37491-500, 2001.

Other inhibitors include FGFR4 binding agents that specifically bind to FGFR4 to block or impair binding of human FGFR4 to one or more ligands, such as FGF2. While such agents bind the receptor, they do not trigger the signaling cascade responsible for FGFR4 activity. Alternatively, soluble FGFR4 receptors may be used to sequester ligands away from FGFR4. In this regard, the extracellular region of FGFR4 can be fused to another moiety to increase serum half-life, e.g., an Fc antibody domain, to make a fusion protein, or to PEG moieties, to generate a soluble FGFR4 receptor.

### Administration Considerations

When treating cancer or modulating cancer cell invasion in vivo, the method is preferably performed as soon as possible after it has been determined that a subject is at risk for cancer (e.g., cancer markers are detected) or as soon as possible after cancer and/or invasion of surrounding tissues is detected (e.g., following tumor resection). To this end, the antibody or fragment thereof is administered before tumor invasion is detected to protect, in whole or in part, against cancer cell invasion, ingrowth, or metastasis. The antibody or fragment thereof also can be administered after tumor invasion has begun to prevent, in whole or in part, further invasion or formation of secondary tumors. In this regard, the invention provides a method of treating a mammalian subject comprising (i) selecting for treatment a mammalian subject diagnosed with or treated for cancer; and (ii) administering to the subject the inventive antibody or fragment thereof (e.g., the inventive antibody or fragment thereof formulated in a composition) in an amount effective to modulate cancer cell invasion, ingrowth, or metastasis.

In preferred embodiments, the antibody or antibody fragment (and/or any other therapeutic agent described herein) is formulated in a composition, such as a physiologically-acceptable composition, comprising a carrier (i.e., vehicle, adjuvant, or diluent). The particular carrier employed is limited only by chemico-physical considerations, such as solubility and lack of reactivity, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers. eds. (1982), and ASHP Handbook on Injectable Drugs, Toissel, 4th ed. (1986)). A pharmaceutical composition comprising any of the materials described herein may be placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

A particular administration regimen for a particular subject will depend, in part, upon the particular antibody used, the presence of other therapeutics, the amount administered, the route of administration, and the cause and extent of any side effects. The amount administered to a subject (e.g., a mammal, such as a human) in accordance with the invention should be sufficient to affect the desired response over a reasonable time frame. The size of the dose also will be determined by the route, timing, and frequency of administration. Accordingly, the clinician may titer the dosage and modify the route of administration to obtain the optimal therapeutic effect, and conventional range-finding techniques are known to those of ordinary skill in the art. Purely by way of illustration, the inventive method can comprise administering, e.g., from about 0.1 μg/kg to up to about 100 mg/kg or more, depending on the factors mentioned above. In other embodiments, the dosage may range from 1 μg/kg up to about 100 mg/kg; or 5 μg/kg up to about 100 mg/kg; or 10 μg/kg up to about 100 mg/kg. Due to the hyperproliferative nature of cancer, a single dose of antibody or fragment thereof may not accomplish a complete anti-cancer (anti-invasive) effect. Indeed, as with most chronic diseases, prolonged treatment involving multiple doses of a therapeutic agent may be required. Accordingly, in one embodiment, the inventive method comprises delivering multiple doses of pharmaceutical composition over a period of time.

Suitable methods of administering a physiologically-acceptable composition, such as a pharmaceutical composition comprising an anti-FGFR4 antibody or fragment thereof, are well known in the art. Although more than one route can be used to administer an agent, a particular route can provide a more immediate and more effective reaction than another route. Depending on the circumstances, a pharmaceutical composition comprising the agent is applied or instilled into body cavities, absorbed through the skin or mucous membranes, ingested, inhaled, and/or introduced into circulation. For example, in certain circumstances, it will be desirable to deliver a physiologically-acceptable (e.g., pharmaceutical) composition through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the antibody or fragment thereof is administered regionally via intraarterial or intravenous administration feeding the region of interest, e.g., via the hepatic artery for delivery to the liver. Alternatively, the composition is administered locally via implantation of a membrane, sponge, or another appropriate material on to which the antibody has been absorbed or encapsulated. Where an implantation device is used, the device may be implanted into any suitable tissue or organ, and delivery of the antibody may be via diffusion, timed-release bolus, or continuous administration. In other aspects, the agent is administered directly to exposed tissue during tumor resection or other surgical procedures or by targeted injection (e.g., intratumoral injection). Therapeutic delivery approaches are well known to the skilled artisan, some of which are further described, for example, in U.S. Pat. No. 5,399,363.

### Combination Therapy

When appropriate, the agent is administered in combination with other substances (e.g., therapeutics) and/or other therapeutic modalities to achieve an additional (or augmented) biological effect. For example, in one embodiment, the inventive method comprises administering two or more different anti-FGFR4 antibodies (or fragments thereof) to a subject. In this regard, when the inventive method entails using an antibody that binds an FGFR4 epitope recognized by mAb F90-10C5, the method may further comprise administering to a subject (or contacting a population of cancer cells with) an antibody or fragment thereof that binds an epitope of FGFR4 that is different than the epitope recognized by mAb F90-10C5. Exemplary second antibodies and fragments thereof include (i) F85-6C5 and F90-3B6 (also referred to herein as “6C5” and “3B6,” respectively), described in the Examples, (ii) antibodies or fragments thereof that compete for binding of FGFR4 with F85-6C5 and/or F90-3B6, and (iii) antibodies or fragments thereof that bind the region of FGFR4 recognized by F85-6C5 and/or F90-3B6. Surprisingly, exposing cancer cells to mAb F90-10C5 in combination with F85-6C5 or F90-3B6 results in a greater reduction in total MT1-MMP protein and activated MT 1-MMP protein in the cells compared to treatment with mAb F90-10C5 alone. Combination treatment with two or more anti-FGFR4 antibodies recognizing different FGFR4 epitopes (especially different extracellular epitopes) can enhance the inhibitory effect of mAb F90-10C5.

Alternatively (or in addition), multiple antibodies are delivered to a subject to obtain multiple biological effects. In one aspect, the invention provides a method of treating a mammalian subject comprising administering to a subject diagnosed with or treated for cancer a first and a second anti-FGFR4 antibody or FGFR4-binding fragment thereof, wherein the first anti-FGFR4 antibody or fragment inhibits FGF2-induced phosphorylation of FGFR4 R388, and the second anti-FGFR4 antibody or fragment inhibits ligand-independent FGFR4 phosphorylation. The antibodies or fragments thereof may be formulated in a single composition, or administered in separate compositions (i.e., a first composition containing the first antibody or fragment and a second composition containing the second antibody or fragment) to be administered simultaneously or sequentially. The inventive method also may entail administering the anti-FGFR4 antibody in combination with a non-antibody based FGFR4 inhibitor, such as those described further herein. For example, the method can comprise administering to the subject a standard of care chemotherapy for cancer.

Alternatively or in addition, the inventive method further comprises administering to a subject (or contacting a population of cancer cells with) an MT1-MMP inhibitor. Any inhibitor of MT1-MMP is suitable for use in the context of the invention, and non-antibody-based (e.g., small molecule) MT1-MMP inhibitors are described above. In one aspect, the inhibitor is an antibody or fragment thereof that binds MT1-MMP to inhibit the enzyme's activity (e.g., inhibit extracellular matrix degradation). Several anti-MT1-MMP antibodies are known in the art. For example, antibodies LEM-1 and LEM-2, further described in Nisato et al., Cancer Res., 65(20): 9377-9387, 2005, inhibit cytokine-induced bovine microvascular endothelial (BME) cell invasion of three-dimensional collagen gels in a dose-dependent manner. Anti-MT1-MMP antibodies also are described in Galvez et al., J. Biol. Chem., 276: 37491-500, 2001. The discussion of antibodies and fragments thereof provided above with respect to FGFR4 antibodies also is relevant to anti-MT1-MMP antibodies.

Endogenous inhibitors of MT1-MMP have been identified and are contemplated for use in the inventive method. For example, once activated, MMPs are specifically inhibited by a group of endogenous tissue inhibitors of metalloproteinases (TIMPs) that bind to the active site, inhibiting catalysis (Nagase et al., supra). MT1-MMP is inhibited by TIMP-2, TIMP-3 and TIMP-4, but not by TIMP-1 (Will et al., J. Biol. Chem., 271: 17119-17123, 1996; Bigg et al., Cancer Res., 61: 3610-3618, 2001). RECK (reversion inducing-cysteine rich protein with Kazal motifs), a GPI anchored glycoprotein, is another inhibitor of MT 1-MMP (Oh et al., Cell, 107: 789-800, 2001). Mice containing mutated RECK are embryonic lethal at E10.5 showing defects in collagen fibrils, the basal lamina, and vascular development—a phenotype that may correlate with excessive MMP activity. Chondroitin/heparan sulfate proteoglycans, testican 1, testican 3, and a splice variant of testican 3, N-Tes, have also been shown to inhibit MT1-MMP (Nakada et al., Cancer Res., 61: 8896-8902, 2001).

Inhibitors of vascular endothelial growth factor receptor-3 (VEGFR-3) or vascular endothelial growth factor receptor-2 (VEGFR-2) also are contemplated for use with the inventive antibody, fragment thereof, polypeptide, or polynucleotide. The inventive method can comprise administering an agent that inhibits VEGF-D or VEGF-C stimulation of VEGFR-3 or VEGFR-2, such as an antibody or antibody fragment that binds to VEGF-C, VEGF-D, or the extracellular domain of VEGFR-3 or VEGFR-2; a soluble protein comprising a VEGFR-3 extracellular domain or fragment thereof effective to bind VEGF-C or VEGF-D; or a soluble protein comprising a VEGFR-2 extracellular domain or fragment thereof effective to bind VEGF-C or VEGF-D. Exemplary agents and methods for modulating VEGFR-3 and VEGFR-2 activity are described in, e.g., U.S. Pat. Nos. 7,034,105 and 6,824,777, U.S. Patent Publication Nos. 2005/0282233 and 2006/0030000; and International Patent Publication Nos. WO 2005/087812 (Application No. PCT/US2005/007742), WO 2005/087808 (Application No. PCT/US2005/007741), WO 2002/060950 (Application No. PCT/US2002/001784), and WO 2000/021560 (Application No. PCT/US1999/023525).

At any given time medical practitioners have one or more “standard of care” therapies that are regarded as appropriate or preferred for a particular cancer, stage of progression, and patient type, for example. The invention includes, as an additional variation, administration/use of standard or care therapies in combination with therapies described herein.

Other therapeutics/co-treatments suitable for use in conjunction with the inventive method include, for example, radiation treatment, hyperthermia, surgical resection, chemotherapy, anti-angiogenic factors (for instance, soluble growth factor receptors (e.g., sflt), growth factor antagonists (e.g., angiotensin), etc.), pain relievers, and the like. Each therapeutic factor is administered according to a regimen suitable for that medicament. This includes concurrent administration (i.e., substantially simultaneous administration) and non-concurrent administration (i.e., administration at different times, in any order, whether overlapping or not) of the inventive antibody or fragment thereof and one or more additionally suitable agents(s). It will be appreciated that different components may be administered in the same or in separate compositions, and by the same or different routes of administration. In this regard, the inventive composition can comprise an antibody or fragment thereof that binds an epitope of FGFR4 that is different than the epitope recognized by mAb F90-10C5 and/or an MT1-MMP inhibitor. Alternatively or in addition, the inventive antibody or fragment thereof can comprise an anti-neoplastic agent (e.g., a radionucleotide) or cytotoxic agent conjugated or attached thereto. For further discussion of radionucleotide-antibody conjugates, see, e.g., Appelbaum et al., Blood, 73(8): 2202, 1989; and U.S. Pat. No. 6,743,411.

Chemotherapy treatment for use in conjunction with the invention employ anti-neoplastic agents including, but not limited to, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), and hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinium coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p″-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

Cytokines that are effective in inhibiting tumor metastasis are also contemplated for use in the combination therapy. Such cytokines, lymphokines, or other hematopoietic factors include, but are not limited to, M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin.

### EXAMPLES

The invention, thus generally described, will be understood more readily by reference to the following examples, which are provided by way of illustration and are not intended to limit the invention.

### Example 1

This Example identifies FGFR4 and other kinase molecules as regulators of tumor cell invasion using an unbiased gain of function kinome screen for MT 1-MMP activity.

To identify kinase molecules that regulate tumor cell invasion, an unbiased gain of function kinome screen for MT1-MMP activity using gelatin zymography was developed. Since MT1-MMP is the most prominently expressed MT-MMP and the main MMP-2 activator in HT-1080 cells (Lehti et al., J. Biol. Chem., 27(10): 8440-48, 2002), MMP-2 activation served as an indirect measure of MT1-MMP activity. A cDNA library encoding ˜93% of all human protein kinases (564 cDNAs encoding 480 separate kinases) (Varjosalo et al., Cell, 133(3): 537-48, 2008) was transiently transfected with FuGENE6 (Roche) into human HT-1080 fibrosarcoma cells plated in 96-well plates at a density of 1×10⁴ cells/well. After transfection, the cells were incubated in complete medium for 24 hours and serum-free medium for an additional 20 hours. Aliquots of conditioned medium were dissolved in non-reducing Laemmli sample buffer and separated by electrophoresis using discontinuous 3.5:10% polyacrylamide gels containing 1 mg/ml gelatin. SDS was removed to permit MMP refolding and autoactivation as described in Lohi et al., Eur. J. Biochem., 239(2): 239-47, 1996. The gels were then incubated at 37° C. for 16 hours and stained with Coomassie Blue in 10% acetic acid, 5% methanol.

The gels were developed via gelatin zymography, and relative protein levels and activation levels of proMMP-2 and proMMP-9 were quantified from the zymographic images. A subset of kinases that induced the greatest relative level of proMMP-2 were selected for a secondary screen. Since MT1-MMP and MMP-9 are both biologically important MMPs commonly implicated in remodeling processes (including invasion), these MMPs could share common regulatory pathways. However, the kinases that enhanced the relative levels of MMP-9, which was detectable only as proenzyme, were mostly distinct from those enhancing MT1-MMP activity and MMP-2 activation.

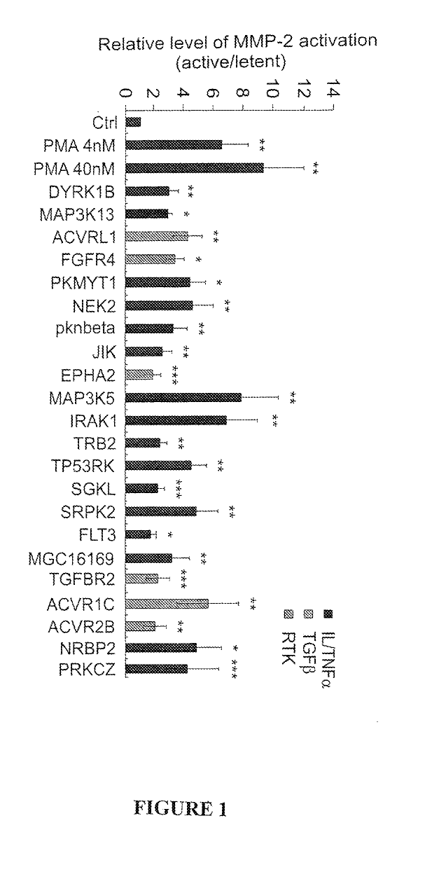

In the secondary MT1-MMP/MMP-2 cascade screen, the expression of 22 kinases prompted a more than 2-fold increase in pro-MMP-2 activation relative to the control (FIG. 1). These kinases included both novel MT1-MMP/MMP-2 cascade regulators and pathway components downstream of known MT1-MMP-inducing stimuli. Among this later group are receptors of TGF-β family members and kinases related to the inflammatory signaling pathways activated by IL-1 or TNF-α. Unexpected hits which significantly increased MMP-2 activation included receptor tyrosine kinases FGFR4 and EphA2.

FGFR4 significantly increased MMP-2 activation, which is an indirect measure of MT1-MMP activity. The results signal FGFR4's role as regulator of tumor cell invasion.

### Example 2

This Example demonstrates that FGFR4's modulation of MT1-MMP activity occurs post-transcriptionally.

Since MT1-MMP gene expression is frequently upregulated in malignant versus normal tissues, the potential contribution of selected kinases on MT1-MMP expression was examined. HT-1080 cells were transfected with expression vectors encoding FGFR4, EphA2, or the most potent kinases in the TGFβ, IL-1, and TNFα pathways. Levels of MT1-MMP mRNA were determined by quantitative real-time PCR. IRAK1, MAP3K13, ACVRIC, and EphA2 moderately but significantly increased the levels of MT1-MMP mRNA (1.5 to 2.5 fold, n=3, p<0.005). Consistent with the in vitro data, correlation blots from the In Silico Transcriptomics (IST) database containing normalized expression data from 8478 malignant samples revealed significant positive correlations between the expression of MT1-MMP and IRAK1, EphA2, or ACVR1L in tissue samples from several types of cancers. Interestingly, FGFR4, which was among the strongest hits in the MT1-MMP/MMP-2 screen, had negligible effects on MT1-MMP mRNA levels in HT-1080 cells. Likewise, only weak correlations between FGFR4 and MT1-MMP expression levels were observed in tissue samples from different types of tumors. This is consistent with independent transcriptional regulation of these two genes and raises the possibility of more direct mechanism of MT1-MMP regulation by FGFR4.

To assess the posttranscriptional effects of kinases on the MT1-MMP levels and subcellular distribution, MT1-MMP was co-expressed with FGFR4, EphA2, or IRAK1 in COS-1 cells that do not express detectable endogenous MT1-MMP. The cells were subjected to immunofluorescence staining with anti-MT 1-MMP antibodies. Upon viewing, MT1-MMP appeared mainly localized in intracellular perinuclear compartments in cells transfected with only MT1-MMP. Interestingly, co-expression of FGFR4 resulted in punctuate MT1-MMP localization in both cytoplasmic and cell surface membrane structures. These alterations coincided with increased cell spreading in FGFR4 expressing cells, which was also seen in EphA2 transfected cells. IRAK increased the intensity of MT1-MMP staining on the cell surface. The catalytic kinase activities were essential for MT1-MMP regulation, since MT1-MMP remained mainly perinuclear in cells expressing mutant kinases with inactivating point mutation in active-site motifs (Varjosalo et al., supra).

The amount of MT1-MMP protein in transfected cells also was studied. MDA-MB-231 human breast cancer cells were transiently transfected with expression vectors encoding various kinases, and the MT1-MMP protein expression assessed by immunoblotting. The cell surface levels of MT1-MMP protein were assessed by Sulfo-NHS-biotin labeling and immunoprecipitation with antibodies against MT1-MMP. FGFR4, IRAK1, and EphA2 all markedly increased the total and cell surface levels of MT1-MMP, although only IRAK1 slightly increased MT1-MMP mRNA expression as assessed by real-time PCR. Both activated MT1-MMP and the autocatalytically processed 43 kDa form, which frequently correlates with high cell surface MT1-MMP activity (Lehti et al., Biochem. J., 334: 345-53, 1998; Lehti et al., J. Biol. Chem., 275: 15006-13, 2000), were detected in IRAK1 and FGFR4 expressing cells. In contrast, MT1-MMP levels were not markedly affected by most nonfunctional kinases.

These results suggest that active FGFR4 post-transcriptionally increases MT1-MMP protein levels and alters its distribution.

### Example 3

This Example demonstrates that FGFR4 and MT1-MMP physically interact and highlights potential mechanisms of MT1-MMP regulation by FGFR4.

To examine possible mechanisms of MT1-MMP regulation by FGFR4, double immunofluorescence staining was performed for FGFR4 or IRAK1 and MT1-MMP in MDA-MB-231 cells transfected with expression vectors encoding the respective kinases. FGFR4-transfected cells were incubated with or without FGF2 (25 ng/ml) for 30 minutes, and then stained with immunofluorescent antibodies against MT1-MMP and FGFR4. IRAK1 transfected cells were incubated with or without IL-1I3 (5 ng/ml) for 30 minutes, and immunofluorescent staining was carried out for MT1-MMP and IRAK1. Interestingly, FGFR4 largely co-localized with MT1-MMP in vesicular membrane structures of untreated cells, and FGF2 stimulation further enhanced this co-localization. In IRAK1 expressing cells, MT 1 -MMP localized prominently on the cell surface with and without IL-1I3 stimulation. Unlike FGFR4, cytoplasmic IRAK staining did not specifically co-localize with MT1-MMP, although both proteins tended to accumulate at the same regions of the cell.

To define whether MT1-MMP and FGFR4, which were located in the same membrane vesicles, would physically interact in the same membrane receptor complexes, the cells were co-transfected to produce HA-tagged MT1-MMP and VS-tagged FGFR4. This was followed by immunoprecipitation and immunoblotting analysis. For immunoblotting experiments, confluent cell cultures were washed and incubated for 24 hours in serum free DMEM. Transiently transfected cells were incubated for 16 hours after transfection before transferring to serum free medium. The conditioned media was then harvested and cell lysates prepared as described (Lehti et al. 1998, supra). SDS-PAGE was carried out using 4-20% gradient Laemmli polyacrylamide gels (Bio-Rad, Hercules, Calif.). The proteins were transferred to nitrocellulose membranes, and their immunodetection was performed as described (Lohi et al., Eur. J. Biochem., 239: 239-47, 1996). The immunoblotting analysis was performed using antibodies against the protein tags, HA and V5.

FGFR4 was clearly detectable by immunoblotting with anti-V5 antibodies in MT1-MMP complexes that had been immunoprecipitated with anti-HA antibodies. Likewise, HA-tagged MT1-MMP was detected in the FGFR4 complexes immunoprecipitated with anti-V5 antibodies. The interaction between FGFR4 and MT1-MMP was specific, since no interactions between V5-tagged IRAK1 and HA-tagged MT1-MMP were detected under the same experimental conditions.

Since FGFR4 has been reported to be mostly recycled after endocytosis, the effects of FGFR4 on endosomal localization of MT1-MMP were assessed. Immunofluorescence analysis with antibodies against endosomal marker proteins (clathrin and EEA1) revealed that MT1-MMP interaction with FGFR4 coincided with the enhanced levels of MT1-MMP in intracellular clathrin and EEA1 positive endosomal vesicles. By contrast, prominent MT1-MMP staining was detected on the surface of IRAK1 expressing cells. These results are consistent with potentially enhanced stability of endocytosed MT1-MMP by interaction with FGFR4.

To define the contribution of lysosomal degradation for the regulation of MT1-MMP activity and protein expression by IRAK1 and FGFR4, MDA-MB-231 cells expressing these kinases were incubated with a lysosomal inhibitor, Bafilomycin A (Calbiochem), and a proteosome inhibitor, MG132 (MG-132, Z-Leu-Leu-CHO; Peptide Institute Inc., Osaka, Japan). MDA-MB-231 cells were transfected with empty pCR3.1 expression vector (mock) and corresponding vectors coding for IRAK1 or FGFR4. Cells were immunostained with anti-MT1-MMP and anti-clathrin antibodies and assessed by confocal imaging. FGFR4 transfected cells were immunostained with anti-MT 1-MMP and anti-LAMP 1 antibodies. The effect of MG132 on the levels of MT1-MMP was marginal. In contrast, the inhibition of lysosomal degradation by Bafilomycin A increased MT1-MMP protein levels in control cells, which is consistent with the reported rapid and constitutive lysosomal degradation of MT1-MMP. Importantly, FGFR4 specifically increased the levels of MT1-MMP in untreated cells, thus decreasing the differences in MT1-MMP protein levels between non-treated and Bafilomycin A treated cells. Accordingly, MT1-MMP localization in LAMP-1 positive lysosomal structures was markedly decreased in FGFR4 expressing cells as compared to control cells.

The results of this Example indicate that FGFR4 inhibits lysosomal sorting and degradation of MT1-MMP, thus increasing the cellular levels of active MT1-MMP. The FGFR4-mediated increase in MT1-MMP, by extension, modulates tumor cell invasiveness.

### Example 4

This Example illustrates the functional significance of kinase-mediated MT1-MMP regulation using a three-dimensional collagen invasion assay, i.e., a tumor cell invasion assay.

Pericellular collagen degradation is the major established biological function of MT1-MMP. Kinase-mediated modulation of MT1-MMP activity was determined in a three-dimensional collagen invasion assay. MDA-MB-231 human breast cancer cells were transfected with expression vectors encoding FGFR4, EPHA2, and IRAK1, or with respective inactivated kinases. The cells were seeded on top of type I collagen gels and allowed to invade for 5 days. The gels were fixed and embedded in paraffin. Cells that invaded into the collagen matrix were visualized and counted from hematoxylin and eosin stained sections.

The overexpression of each of the active kinases significantly increased the otherwise relatively slow invasion of unstimulated MDA-MB-231 cells. The expression of IRAK1, FGFR4, or EphA2 each resulted in over four-fold increased rates of invasion compared to the mock transfected cells. As expected, inactivated kinases had negligible effects on cell invasion. Noteworthy, only FGFR4 and EphA2 significantly increased the number of cells that invaded over 20 μm (12.1 and 9.8 fold, respectively). FGFR4 increased by 20-fold the number of MDA-MB-231 cells that invaded greater than 100 ᵢlm. MT1-MMP and FGFR4 colocalized at both the leading edge and in intracellular vesicles of the rapidly invading cells.

In addition to studying cell invasion, matrix degradation was examined. Transfected cells were seeded on Alexa 488 gelatin coated coverslips and allowed to attach and spread in the presence of GM6001 (10 μM) for three hours. After washing out the MMP inhibitor, cell-mediated gelatin degradation was carried out for 20 minutes in complete medium. Fixed and permeabilized cells were immunostained with anti-MT1-MMP antibodies. Degradation was visualized by confocal microscopy and quantified as the ratio between degradation area and cell number from low magnification figures (mean ±1 SD, n=3). In addition, transfected cells were incubated on crosslinked collagen matrices for three hours and immunostained for MT1-MMP and FGFR4.

Consistent with the higher rates of collagen invasion, cells expressing IRAK1, FGFR4 and EphA2 were also able to degrade fluorescent gelatin substrate more efficiently than the control cells or cells transfected with KD kinases. Interestingly, FGFR4 expression in the cells led to markedly polarized foci of pericellular matrix proteolysis that colocalized with MT1-MMP clusters in one edge of the cells. FGFR4 expressing cells were also able to degrade and traverse crosslinked collagen matrix within 3 hours. In contrast, IRAK1, which efficiently increased gelatin degradation and the levels of MT1-MMP at cell-matrix adhesions, failed to increase the amount of degraded holes in layers of crosslinked 3-D collagen within the same time period.

These results suggest a specific role for FGFR4 in inducing highly invasive cancer cell phenotype, where MT1-MMP functions coordinately with the cellular motile machinery to drive invasion in cross-linked 3-D collagen.

### Example 5

This Example compares the effects of two FGFR4 alleles on MT1-MMP function and cell invasion and establishes FGFR4 as a novel target for inhibiting cancer cell invasion.

A single nucleotide polymorphism in the FGFR4 gene has been linked to poor prognosis in patients with several types of tumors such as breast, prostate, and colon adenocarcinomas as well as head and neck squamous cell carcinomas, melanomas, and soft-tissue sarcomas. In the corresponding FGFR4 variant, glycine 388 in the transmembrane domain is changed to arginine (R388). Interestingly, sequence analysis revealed that the FGFR4 cDNA that was included in the kinome library described in Example 1 encoded the Arg388 variant. To compare the effects of the G388 FGFR4 allele and the R388 FGFR4 allele on MT1-MMP function and cell invasion, cDNA encoding the R388 FGFR of the kinome library was modified to encode wild-type G388 FGFR4. MDA-MB-231 cells were transiently transfected with expression vectors encoding the FGFR4 alleles (FGFR4G388-V5-His and FGFR4R388-V5-His) and corresponding non-functional kinases, as well as expression vectors encoding HA-tagged MT1-MMP. Cells were incubated with Bafilomycin A or GM6001 for 16 hours, and the levels of MT1-MMP and FGFR4 were assessed by immunoblotting. Cell media was harvested and cell lysates prepared as described (Lehti et al. 1998, supra). SDS-PAGE was carried out using 4-20% gradient Laemmli polyacrylamide gels (Bio-Rad, Hercules, Calif.). The proteins were then transferred to nitrocellulose membranes, and their immunodetection was performed as described (Lohi et al., supra).

Lysosomal degradation inhibition by Bafilomycin A in control MDA-MB-231 cells increased MT1-MMP protein levels. The expression of the FGFR4 R388 variant relatively decreased this effect by increasing the levels of MT1-MMP in non-treated cells. In contrast, MT1-MMP levels were not markedly increased by the expression of FGFR4 G388. In FGFR4 G388 expressing cells, the increase in MT1-MMP levels after Bafilomycin treatment was coupled with considerable FGFR4 down-regulation. The effect was less apparent in cells expressing R388 or either one of the inactivated kinases. Inhibition of MT1-MMP activity by GM6001, a synthetic MMP inhibitor, correlated with detection of endogenous FGFR4 in control cells that normally express FGFR4 G388 at levels undetectable by immunoblotting.

The opposite effects mediated by FGFR4 G388 and FGFR4 R388 on MT1-MMP protein expression also were evident in co-transfection experiments. FGFR4 G1y388 expression was coupled with decreased MT1-MMP protein levels, while the FGFR4 Arg388 variant enhanced the relative levels of MT1-MMP. FGFR4 Arg388 co-precipitated prominently with MT1-MMP. Both alleles and the inactive kinase mutants were, however, detectable in MT1-MMP immunoprecipitates.

The effect of the two FGFR4 alleles on cell invasion also was examined using methods similar to those described in Example 4. cDNA coding for the G388 allele was stably expressed in MDA-MB-231 cells, which were plated atop type 1 collagen gels. While FGFR4 R388 expressing cells invaded collagen at increased rates compared to mock transfected cell, the FGFR4 G388 expressing cells invaded at the same rate as, or even more slowly than, the mock transfected cells. Collagen invasion was abolished by lentiviral MT1-MMP silencing RNA, confirming the functional link between MT1-MMP and FGFR4 R388 in driving the invasion.

This Example demonstrates that FGFR4 G1y388 and MT1-MMP are reciprocally downregulated through a mechanism that depends on the respective kinase and metalloproteinase activities. Therefore, G1y388 and/or Arg388 FGFR4 variants are targets for inhibiting tumor cell invasion.

### Example 6

The results of this Example establish that FGFR4 and MT1-MMP are co-expressed in invasive cancer cells in vivo, further supporting a functional link between the molecules.

The mRNAs for MT1-MMP and FGFR4 are frequently co-expressed in samples from different types of human cancers. While expression in individual tissue samples do not correlate well, the mean expression levels of both MT1-MMP and FGFR4 are frequently upregulated in different tumor types including colon, testis, uterus and breast carcinomas. These results suggest that, when both proteins are expressed in the same cells in vivo, their interaction functionally contributes to the invasiveness of human tumors. To further examine co-localization of MT1-MMP and FGFR4 in different tumor and stromal cell populations, frozen tissue arrays containing 40 malignant and normal breast tissue samples were obtained and immunostained with anti-MT1-MMP and anti-FGFR4 antibodies. The anti-FGFR4 antibodies were well established polyclonal antibodies that do not cross-react with other FGFRs in cultured cells. Monoclonal anti-MT1-MMP antibodies produced by immunization of an MT1-MMP-/- mouse (Ingvarsen et al., Biol. Chem., 389: 943-53, 2008) were used. When tested in immunofluorescence staining of MDA-MB-231 breast cancer cells, the anti-MT1-MMP antibodies readily detected endogenous MT1-MMP, while the staining was completely blocked after siRNA mediated MT1-MMP knock-down. The specimens were analyzed with a Leica microscope.

FGFR4 was observed to be localized predominantly to breast epithelial and carcinoma cells. FGFR4 was detected in ductal epithelial cells in all four cases of normal breast analyzed, and the relative levels were frequently increased in ductal carcinoma cells (strong staining in 20/36 cases). MT1-MMP was significantly upregulated in the reactive stroma in breast carcinomas (28/36 cases), especially in the myoepithelium adjacent to the carcinoma cells. This was observed both in invasive and noninvasive areas of the carcinomas. Importantly, MT1-MMP was specifically detected in the carcinoma cells that were located in the invasive fronts of tumors and in cells of poorly differentiated breast carcinomas (10/36 cases), where MT1-MMP prominently co-localized with FGFR4. Immunohistochemistry of multiple frozen tissue arrays with samples from 14 different tumor types and corresponding normal tissues revealed that the relative levels of MT1-MMP staining were also enhanced in most other malignant tissues (10/14 cases). MT1-MMP was frequently upregulated in the reactive stroma (6/14 cases) and co-expressed with FGFR4 in tumor cells (8/14 cases). As with breast carcinomas, MT1-MMP in other types of carcinomas, such as colon adenocarcinomas, was prominently expressed in the tumor cells at the invasive fronts.

Expression of FGFR4 alleles (FGFR4 G388 and FGFR4 R388) and MT1-MMP in vivo was examined using qPCR array coupled with FGFR4 sequencing from 48 human breast cancer cDNA samples. RNA was extracted with RNeasy Mini Kit (Qiagen) followed by reverse transcription with random hexamer primers (Invitrogen) and Superscript II reverse transcriptase (Life Technologies). mRNA expression was quantified as described (Tatti et al., Exp. Cell Res., 314: 2501-2514, 2008) using TaqMan Universal PCR Master Mix and validated primers (MT1-MMP; Hs 01037006 gH, MT2-MMP; Hs 00233997 ml, MT3-MMP; Hs 00234676 ml, MT4-MMP; Hs 00211754m1, MT5-MMP; Hs 00198580m1, MT6-MMP; Hs 00360861 ml; MMP-9; Hs00957555 ml; FGFR1; Hs00915140 ml, FGFR4; Hs00242558 ml (Applied Biosystems)). The expression was normalized with TATA-binding protein (TBP) and glyceraldehyde 3-phosphate dehydrogenase (GAPDH) mRNA expression. The fragments of FGFR4 cDNA containing the Gly388 to Arg388 site (base pairs 1329-1331) were amplified by PCR (primers: TACCAGTCTGCCTGGCTC (SEQ ID NO: 17) and AGTACGTGCAGAGGCCTT (SEQ ID NO: 18)) and digested with BstN1 (New England BioLabs). The R388 allele was identified by a specific 126 base pair fragment. The G388 allele was identified by two fragments (97 and 29 base pairs).

Of the 48 human breast cancer cDNA samples, 22 had homozygous G/G (46%); 22 had heterozygous G/R (46%); and four had homozygous R/R (8%) alleles of FGFR4. Consistent with the poor prognosis reported in cancer patients, all four cDNA samples with homozygous R388 alleles were from the highest grade (3) breast carcinomas. MT1-MMP mRNA was prominently expressed in all of these tumors co-incident with a lower FGFR4 R388 expression.

The observations described in this Example strongly suggest that FGFR4 and MT1-MMP have a synergistic effect on tumor invasiveness, and confirm that the FGFR4 R388 allele strongly correlates with highly invasive cancers.

### Example 7

This Example illustrates the functional significance of FGFR4 R388 activation and FGFR4 G388 suppression in prostate carcinoma cell invasion of collagen.

Since MT1-MMP and FGFR4 were most frequently co-expressed in human prostate cancers, corresponding cell lines were assessed for endogenous MT1-MMP and FGFR4 expression. PC3 and DU145 prostate adenocarcinoma cells expressed notable levels of FGFR1, MT1-MMP, and either the R388 (PC3 cells) or G388 (DU145 cells) variant of FGFR4. Notably, only PC3 cells expressing a homozygous FGFR4 R388 allele invaded collagen gels efficiently, while the corresponding DU145 cells homozygous for the FGFR4 G388 allele did not. FGFR4 siRNAs inhibited 87% of the PC3 cell invasion, which also was essentially blocked by TIMP-2 -mediated MT1-MMP inhibition. Consistent with the reported induction of proliferation and motility through both FGFR1 and FGFR4 (Sahadevan et al., J. Pathol., 213: 82-90, 2007), siRNAs transiently targeting either one of these FGFRs reduced FGF2-induced ERK phosphorylation. However, when FGFR1 mRNA expression was silenced, collagen invasion increased, suggesting that FGFR1 has a distinct function. Notably, although the DU145 cells expressed less MT1-MMP mRNA, these cells also invaded collagen after transfection with FGFR4 R388.

Cell growth also was examined. Stable MT1-MMP or FGFR4 knockdown by lentiviral shRNAs did not significantly alter the normal monolayer growth of either PC3 or DU145 cells. However, when the cells were plated inside a growth-restricting 3-D matrix composed of cross-linked collagen I, MT1-MMP silencing significantly decreased PC3 and DU145 cell growth and invasion. Likewise, FGFR4 R388 knockdown inhibited the growth and invasion of PC3 cells, as well as decreasing MT1-MMP content and fibroblast receptor substrate-2 (FRS2) phosphorylation. In contrast, knockdown of FGFR4 G388 in DU145 cells increased invasive properties of those cells in collagen coincident with increased levels of endogenous MT1-MMP. FRS2 and ERK phosphorylation also increased in stable FGFR4 G388 and MT1-MMP knockdown cells. Even strong inhibition of FRS2 phosphorylation in PC3 cells was not associated with ERK activation, suggesting that pathways other than FGFR4 activation are involved in mitogenic FGF signaling.

Altogether, the results described above identify FGFR4 R388 as a novel co-factor in MT1-MMP-driven PC3 tumor cell invasion and growth in 3-D collagen, and suggest that the G388 and R388 alleles have opposite effects on MT1-MMP-dependent invasion.

### Example 8

This Example demonstrates that FGFR4 R388 induces MT1-MMP phosphorylation and endosomal stabilization, highlighting an additional potential mechanism of MT1-MMP regulation by FGFR4.

MT1-MMP cytoplasmic tail contains a single tyrosine residue that can be phosphorylated by Src (Nyalendo et al., J. Biol. Chem., 282: 15690-15699, 2007). To determine if FGFR4 can induce MT1-MMP phosphorylation, FGFR4 G388 and FGFR4 R388 were co-expressed with HA-tagged MT1-MMP followed by MT1-MMP immunoprecipitation. MT1-MMP tyrosine phosphorylation was repeatedly detected in COST cells co-expressing MT1-MMP with either allele of FGFR4, but not in cells expressing FGFR4 protein having non-functional kinase domains or only MT1-MMP. FGFR4 R388 and MT1-MMP co-localized mainly in intracellular vesicles. FGF2-treatment increased FGFR4 R388 autophosphorylation, as well as MT1-MMP phosphorylation and endosomal accumulation, suggesting that activated FGFR4 R388 induces MT1-MMP phosphorylation in the complexes. This interaction increased the stability of endocytosed MT1-MMP, as indicated by enhanced co-localization with early endosomal antigen-1 (EEA1) and clathrin, and reduced co-localization with lysosome-associated membrane protein-1 (LAMP1). In contrast, the degree of co-localization of MT1-MMP with the weakly/transiently activated FGFR4 G388 or the kinase-deficient KD proteins in the intracellular vesicles was significantly lower than that observed with FGFR4 R388.

MT1-MMP's only intracellular tyrosine residue was mutated to phenylalanine (MT1-Y/F) to assess the significance of MT1-MMP phosphorylation. The Y573F mutation did not appear to alter MT1-MMP-activity in HT-1080 cells that express very little endogenous FGFR4. However, the mutation abrogated the co-localization of wild-type MT1-MMP and endogenous FGFR4 R388 in cell-cell contacts and intracellular vesicles of MDA-MB-231 cells. MT1-Y/F was localized predominantly at the cell surface, while FGFR4 R388 translocated into intracellular vesicles. Consistent with enhanced cell-surface MT1-MMP, the mutation enhanced cell growth and invasion in 3-D collagen. Accordingly, fewer FGFR4 R388/MT1-Y/F complexes were observed compared to FGFR4 R388/MT1-MMP complexes in transfected MDA-MB-231 cells. While the FGFR4 R388 levels were slightly decreased, FGFR4 G388 protein and FGFR4 G388/MT1-Y/F complexes were sufficiently suppressed to be undetectable in cells with high MT1-Y/F content. Consistent with the increased detection of endogenous FGFR4 G388 after MT1-MMP inhibition, overexpressed FGFR4 G388 also was suppressed by wild type MT1-MMP, but not by mutant MT1-E/A in which proteinases activity is abrogated.

The observations described above suggest that FGFR4 suppression by unphosphorylated MT1-MMP provides FGFR4 G388-containing cells a feedback mechanism to sustain proinvasive MT1-MMP activity. While MT1-MMP did not alter the phosphorylation of FGFR4 G388, FGFR4 R388 phosphorylation was markedly enhanced by MT1-MMP, but not an inactive MT1-MMP mutant. The interactions, phosphorylation, and trafficking of MT1-MMP/FGFR4 R388 complexes thus seem to support their synergistic functions in cell invasion.

### Example 9

This Example demonstrates that endogenous FGFR4/MT1-MMP activity controls tumor growth and invasion in vivo.

To determine if targeting of the FGFR4/MT1-MMP axis regulates tumor cell behavior in vivo, tumor growth, morphology, and extracellular matrix (ECM) composition were analyzed after subcutaneous injection of PC3 and DU145 cells into SCID mice. PC3 and DU145 cells were lentivirally transduced with a renilla luciferase-green fluorescent protein (GFP)-fusion reporter protein. Stable cell pools expressing scrambled, MT1-MMP, and FGFR4-targeting short-hairpin-RNAs were produced by lentiviral transduction followed by puromycin (Sigma) selection. Greater than 90% knockdown efficiencies were confirmed by qPCR. The cells (2×10⁶) were implanted into the abdominal subcutis of ICR-SCID male mice (5-7 wks of age; Taconic) and allowed to grow for 6-8 weeks. Tumor size was measured with a caliper and noninvasive bioluminescence, which was visualized after intraperitoneal injection of 35 μg/100 μl coelentetrazine using a Xenogen IVIS System (Xenogen).

Stable silencing of either MT1-MMP or FGFR4 R388 dramatically decreased the growth rates of PC3 tumors and the number of stromal vessels containing intravasated tumor cells. A fibrous capsule accumulated around the tumor, and intratumoral extracellular matrix (ECM) separated the MT1-MMP and FGFR4 R388 knock-down tumor cells into small compartments that showed decreased rates of proliferation. The mitotic index, growth, invasion, and metastasis of the tumors correlated inversely with collagen and other ECM protein content. At the same time, cells exhibited increased polarization towards collagen IV, fibronectin and laminin, and increased acinar lumen formation was detected.

Consistent with observations from in vitro assays, MT1-MMP silencing also reduced the growth of DU145 tumors while increasing collagen content. In MT1-MMP knockdown DU145 tumors, FGFR4 G388 mRNA expression increased between two and four-fold, suggesting that a transcriptional feedback mechanism was involved in FGFR4 G388 suppression by MT1-MMP. In contrast, FGFR4 G388 silencing produced more pronounced invasion and extravasation of the DU145 tumor cells while reducing collagen accumulation inside the tumors and at the tumor edge. No significant changes were detected in collagen mRNA expression by qPCR.

The results described above demonstrate that silencing either component of the MT1-MMP/FGFR4 R388 complex inhibited tumor growth, invasion and metastasis. While not wishing to be bound to any particular theory, the inhibition appeared to result from blocking the proteolytic degradation of ECM that physically restricts tumor spread and promotes epithelial differentiation. Silencing FGFR4 G388 achieved the opposite effect.

### Example 10

This Example provides an exemplary method of generating anti-FGFR4 monoclonal antibodies, such as the antibodies of the present invention. The Example also provides a method of characterizing the binding affinity of an anti-FGFR4 antibody or fragment thereof.

A baculovirus expression vector encoding the extracellular region of FGFR4 linked to a His-tag was constructed according to methods standard in the art. Recombinant baculoviruses were generated by co-transfection of Sf9 cells with a recombinant FGFR4 ectodomain coding vector and linearized BACULOGOLD™ DNA (Pharmingen). High Five cells were infected with the viral stocks obtained from Sf9 cells, and the recombinant His-tagged FGFR4 protein was purified using nickel columns for immunization. Hybridoma clones were generated using standard methods and subcloned as required. Ascites fluids or culture medium from cultured hybridoma cell clones were screened by immunoblotting using recombinant FGFR4 ectodomain/Fc-fusion protein (R&D systems). Positive clones were further assessed by immunoblotting using lysates of control and FGFR4-transfected COS-7 cells, as well as by immunofluorescence of corresponding cells. Monoclonal antibodies were purified using HiTrap Protein G columns according to the instructions (GE Life Sciences). Three monoclonal antibodies, F85-6C5, F90-3B6 and F90-10C5, were subjected to function blocking analysis.

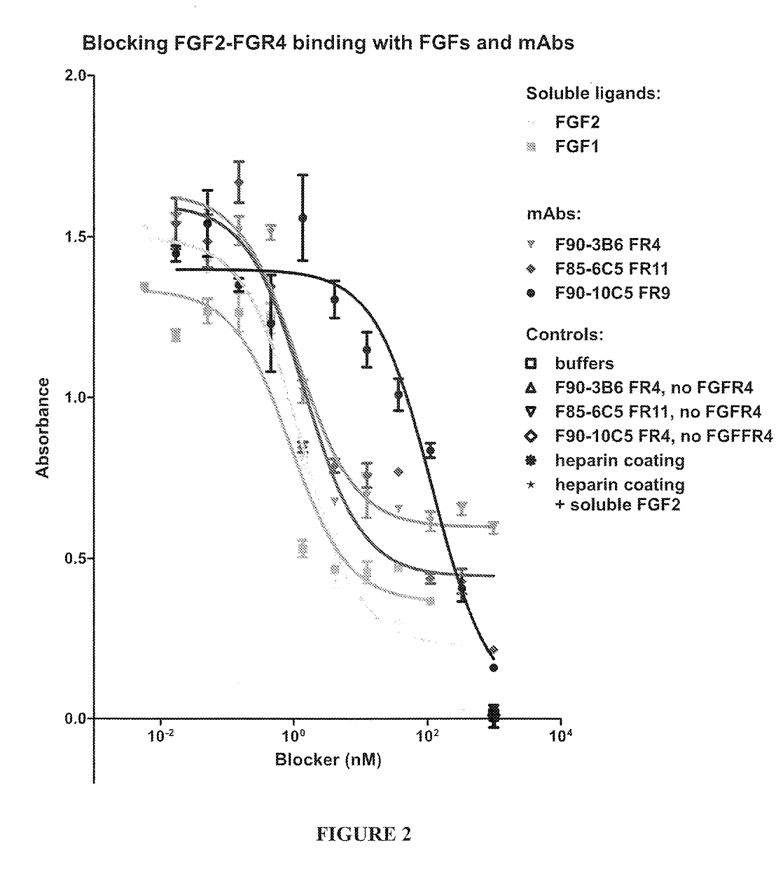

The affinities of the mAbs 3B6, 6C5 and 10C5 for FGFR4-Fc were compared using a receptor binding assay in the enzyme linked immunoassay format (FIG. 2). Recombinant human FGFR4-Fc, comprising the FGFR4 extracellular domain (amino acid residues 1-369, Partanen et al., Proc. Natl. Acad. Sci. USA, 87: 8913-8917, 1990) fused to the carboxyterminal region of human IgG (amino acid residues 100-330) via a polypeptide linker, was obtained from R&D Systems (Cat # 685-FR). Microtiter plate wells (ThermoElectron, Cat #95029100) were coated with recombinant human FGF2 (R&D Systems, Cat# 233-FB) and heparin (Sigma-Aldrich, Cat# H-3149) in 0.1M NaHCO3, pH 9.5. The wells were washed (100 mM Tris, 150 mM NaCl, 0.1% (v/v) Tween 20, pH 7.5) and available non-specific protein binding sites were blocked with PBS, 0.05% (v/v) Tween 20, 0.5% BSA. The wells were washed a second time. FGFR4-Fc was preincubated with a dilution series of each mAb (3B6, 6C5 or 10C5) in 100 mM Tris, 150 mM NaC1, 0.1% Tween 20, 1% BSA, 0.1 μg/ml heparin, pH 7.5, before competitive binding to the FGF2-heparin-coated wells. After addition of preincubated FGFR4-Fc and further incubation, the FGF2 coated wells were washed. Bound FGFR4-Fc was detected using goat anti-human alkaline phosphatase conjugate (Sigma-Aldrich, Cat# A9544).

As shown in FIG. 2, the potencies of the mAbs 3B6, 6C5 and 10C5 for blocking the binding of FGFR4-Fc to immobilized FGF2 differed. Maximal FGFR4-FGF2 interaction is seen with low concentrations of the blockers, while at higher concentrations the interaction (measured in absorbance units) is inhibited to the level of background absorbance. The mAbs 3B6 and 6C5 blocked the binding of FGFR4 to immobilized FGF2 with similar potencies as soluble FGF2 or FGF1. Also, the half-maximal inhibitory concentrations (IC₅₀) of mAbs 3B6 and 6C5 were close to those of FGF2 and FGF1 (1.077 nM, 0.3019 nM, 0.6914 nM, and 0.7334 nM, respectively). mAb 10C5 blocked FGFR4-FGF2 interaction more weakly than the two other mAbs, as indicated by a shift of the blockage curve toward higher concentrations of blocker and from a higher half-maximal inhibitory concentration (6.217 nM). The binding of FGFR4 to immobilized FGF2 was specific, indicated by a very low absorbance level obtained from heparin coated wells (after preincubation with soluble FGF2 or without soluble ligand). Use of the mAbs without prior addition of FGFR4 provided absorbance values at background level. Thus, the assay measures the blockage of FGFR4-FGF2 binding specifically.

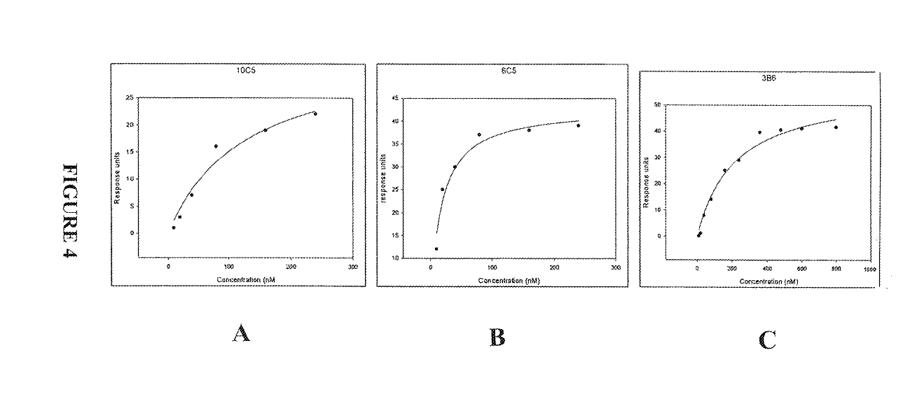

Differential binding of mAbs 3B6, 6B5, and 10C5 to a FGFR4-Fc fusion protein immobilized on a biosensor chip were measured in BIAcore assays, i.e., surface plasmon resonance using a biosensor (BIAcore 2000®, BIAcore AB). FGFR4-Fc was diluted in 10 mM sodium acetate buffer, pH 4.7, and amine-coupled to a BIAcore sensor chip. The amount of immobilized FGFR4-Fc used generated a 1000 response unit signal when saturated with the anti-FGFR4 monoclonal antibodies. An uncoupled biosensor chip channel was used to measure unspecific background signal, which was subtracted from the signal obtained from the FGFR4-Fc coupled channel. A dilution series of each mAb was injected over FGFR4-Fc on a biosensor chip and binding measured in relative response units. Each mAb (3B6, 6C5 or 10C5) was injected at 10 nM to 240 nM in PBS buffer. The flow rate was maintained at 20 μl/min and a 5 minute binding phase was used. Following mAb injection, the flow was exchanged with PBS buffer to determine the rate of dissociation. The sensor chip was regenerated between cycles with a 30 second pulse of 10 mM glycine, pH 2.2. Kinetics were analyzed by 1:1 Langmuir fitting using BIAcore evaluation software 3.1. For comparison, Kd values were also estimated by plotting the maximal relative response units obtained with a dilution series of each mAb. FIGS. 4A-C show the concentration of the mAb on the X-axis and the obtained response units on the Y-axis. The dissociation equilibrium constant (Kd) of each mAb was estimated, after curve fitting, by the concentration half-way between the obtained maximal and minimal response units. Based on these Kd values, the affinity of mAb 6C5 to immobilized FGFR4-Fc (Kd 1.8×10⁻⁸ M) is significantly higher than that of mAb 3B6 or mAb 10C5 which have similar affinities (Kds 2.17×10⁻⁷ and 1.33×10⁻⁷ M, respectively).

In addition, the FGFR4 epitope recognized by F90-10C5 was identified. A PepSpot array composed of a series peptides covering the amino acid sequence of the extracellular domain of FGFR4 (excluding signal sequence) was obtained. The peptides were 15 amino acids in length and comprised sequences that overlapped by three amino acids. An immunoblotting assay was performed using the monoclonal anti FGFR4 antibodies described above. mAb 6C5 and 3B6 did not recognize linear epitopes, whereas 10C5 detected the following peptides: YKEGSRLAPAGRVRG (SEQ ID NO: 5); GSRLAPAGRVRGWRG (SEQ ID NO: 6); LAPAGRVRGWRGRLE (SEQ ID NO: 7); AGRVRGWRGRLEIAS (SEQ ID NO: 8); and VRGWRGRLEIASFLP (SEQ ID NO: 9). The peptide comprising SEQ ID NO: 7 prompted the strongest signal. The location of SEQ ID NOs: 5-9 in the extracellular region of FGFR4 is illustrated in FIGS. 3A-3C.

Hybridoma 10C5, which produces antibody F90-10C5, was transferred to Deutsche Sammlung von Mikroorganismen and Zellkulturen GmbH (DSMZ), Mascheroder Wep lb. D-38124, Germany, on Sep. 2, 2008, under the provisions of the Budapest Treaty for the International Recognition of the Deposit of Microorganisms for the Purpose of Patent Procedure (“Budapest Treaty”), and assigned Deposit Accession No. DSM ACC2967 on Sep. 4, 2008. Hybridomas F85-6C5 and F90-3B6, which produce mAb F85-6C5 and F90-3B6, respectively, also were deposited with DSMZ under the provisions of the Budapest Treaty, and were assigned Deposit Accession Nos. DSM ACC2966 (F85-6C5) and DSM ACC2965 (F90-3B6) on Sep. 4, 2008.

### Example 11

This Example examines the contribution of FGFR4 G388 and FGFR4 R388 to collagen invasion, and describes inhibition of MT1-MMP-mediated cancer cell invasion using a monoclonal anti-FGFR4 antibody.

Given the distinct effects of the two FGFR4 variants on the levels of MT1-MMP, their contribution to collagen invasion was determined. MDA-MB-231 cells were transfected with an expression vector encoding either FGFR4 G388 or the FGFR4 R388 variant. A portion of the transfected cells was treated with 10 μg/ml control IgG or the monoclonal anti-FGFR4 antibodies 3B6, 6C5 and 10C5. The transfected cells were plated on 3-D collagen in a dual chamber apparatus. FGF2 (25 ng/ml) was used as a chemoattractant to stimulate invasion. The cells were allowed to invade for 5 days before quantification of the invasive foci.

FGFR4 R388-expressing MDA-MB-231 cells invaded at higher rates than either mock transfected or FGFR4 G388 expressing cells. Invasion of control cells, FGFR4 G388-expressing cells, and FGFR4 R388-expressing cells was completely blocked by reducing MT1-MMP mRNA (85% reduction) using lentiviral shRNA against MT1-MMP (Open Biosystems, Huntsville, Ala.). These results further support the identified functional link between FGFR4 and MT1-MMP in cancer cell invasion.

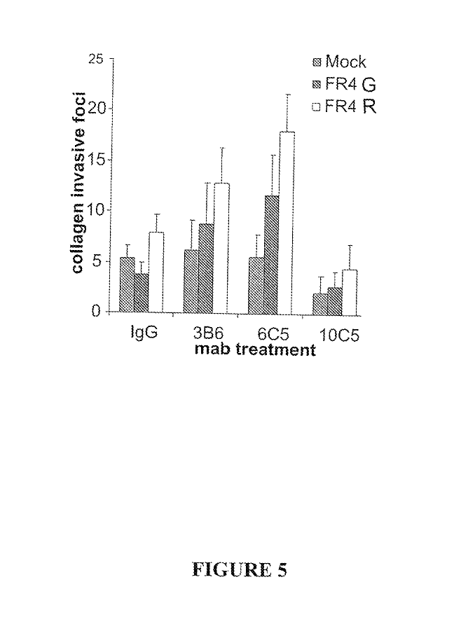

Monoclonal antibodies F85-6C5, F90-3B6, and F90-10C5, which compete for ligand binding to FGFR4, were added to both the upper and lower chamber of the dual chamber collagen assay. FGFR4 R388-induced invasion was efficiently inhibited by the 10C5 monoclonal anti-FGFR4 antibody (FIG. 5). In contrast, the 3B6 and 6C5 antibodies tended to enhance the invasion of both FGFR4 G388 and R388 expressing cells (FIG. 5).

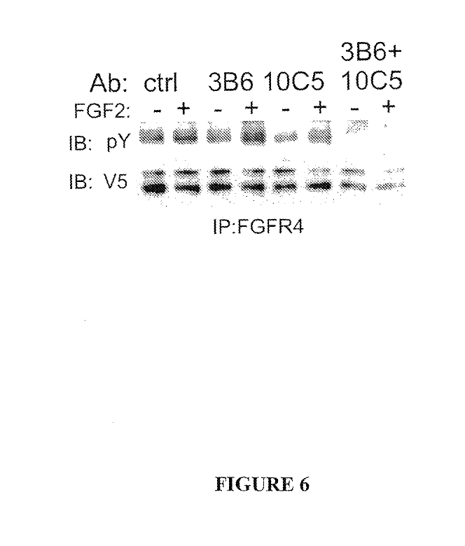

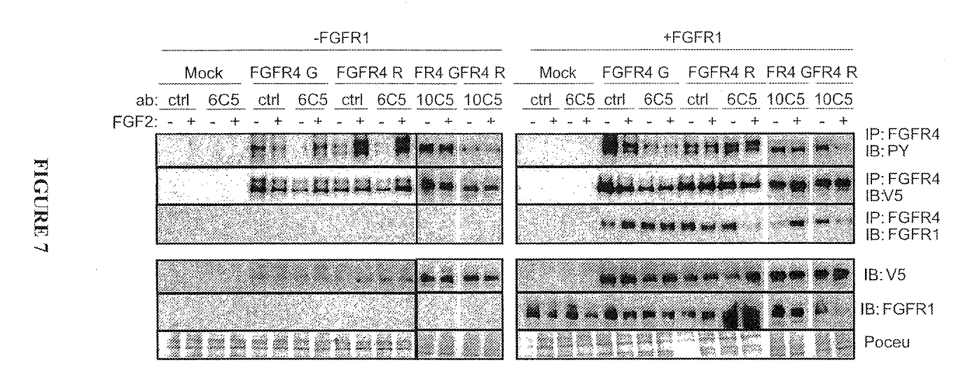

FGFR4 activation in the presence of anti-FGFR4 antibodies was examined to elucidate possible mechanisms behind inhibition of cell invasion. Serum-starved MDA-MB-231 cells expressing FGFR4 R388 or FGFR4 G388 (both tagged with V5) were pretreated with F85-6C5, F90-3B6, and F90-10C5 antibodies (10 μg/ml) overnight and left unstimulated or incubated with FGF2 (10 ng/ml) for 15 minutes. The cell extracts were subjected to immunoprecipitation with antibodies against FGFR4, followed by immunoblotting using antibodies against V5, phosphotyrosine residues, FGFR1, or phosphorylated forms of ERK1/2. The immunoblot is depicted in FIG. 6. Separately, COS-1 cells were transfected to express V5-tagged FGFR4 G388, V5-tagged FGFR4 R388, and FGFR1 alone or in combination. After serum starvation, the cells were pretreated with the anti-FGFR4 antibodies (10 μg/ml) for 30 minutes. A portion of the transfected cells were left unstimulated, while others were incubated with FGF2 (10 ng/ml) for 15 minutes. FGFR4 proteins were immunoprecipitated and immunoblotted with anti-phosphotyrosine and anti-V5 antibodies. The immunoblot is depicted in FIG. 7.

Interestingly, FGFR4 G388 was prominently autophosphorylated in the presence and absence of ligand stimulation, whereas phosphorylation of the FGFR4 R388 variant was highly increased after incubation with FGF2. Treatment of FGFR4 G388 expressing cells with invasion promoting F85-6C5 antibodies (i) suppressed ligand-independent FGFR4 autophosphorylation but (ii) enhanced FGF2-induced phosphorylation. F85-6C5 antibodies modestly suppressed ligand-independent autophosphorylation of FGFR4 R388, whereas ligand-stimulated phosphorylation was not notably affected. Of note, the phosphorylation patterns of mAb 6C5 treated cells expressing either FGFR4 variant were analogous. Treatment of cells expressing FGFR4 R388 with mAb F90-10C5 reduced ligand-independent and ligand-induced phosphorylation of the protein. Phosphorylation was further reduced when cells were exposed to both mAb 10C5 and mAb 3B6, which binds a different FGFR4 epitope compared to mAb 10C5 (FIG. 6).

Since MDA-MB-231 cells express high levels of endogenous FGFR1, the potential effects of FGFR4 expression on total FGFR1 levels and the activation of downstream ERK pathway was analyzed. In both control and FGFR4 R388 expressing cells, FGF2 slightly increased ERK1/2 phosphorylation without notably affecting FGFR1 levels. In contrast, FGFR1 levels were markedly decreased in FGFR4 G388 expressing cells coincidentally with the suppression of FGF2 induced ERK1/2 phosphorylation. Interestingly, the treatment of FGFR4 G388 expressing cells with mAb 6C5, but not with invasion-blocking 10C5 antibodies, rescued both FGFR1 levels and ERK activation after FGF2 stimulation. This is consistent with functional co-operation between FGFR1 and FGFR4 that may contribute to tumor cell invasion and be affected by the invasion-modulating anti-FGFR4 antibodies.

These results were confirmed using transfected COS-1 cells which do not naturally express FGFRs. FGFR4 G388 was prominently autophosphorylated in the absence of ligand stimulation, whereas the phosphorylation of R388 variant was highly increased after 15 minutes incubation with FGF2. The treatment of FGFR4 G388 expressing cells with invasion promoting mAb 6C5 resulted in suppression of ligand-independent FGFR4 autophosphorylation and enhanced FGF2 induced phosphorylation. Modest ligand-independent autophosphorylation of FGFR4 R388 was also suppressed by mAb 6C5, whereas the ligand-stimulated phosphorylation was not notably affected. Antibody 6C5 also reduces FGFR4/FGFR1 heterodimerization following FGF2 stimulation. Of note, the phosphorylation patterns of mAb 6C5 treated cells expressing either FGFR4 variant were analogous. Consistent with the predicted functional FGFR1/FGFR4 interaction, co-expression of these receptors resulted in markedly increased ligand-independent phosphorylation of FGFR4 G388 and R388. This was not notably affected by mAb 6C5. mAb 6C5 may exert its effects on cell invasion through inhibiting constitutive FGFR4 autophosphorylation, and leaving more FGFR4 available for heterotypic interactions with FGFR1 or ligand-induced homotypic FGFR4 signaling. Treatment with mAb 10C5 resulted in inhibition of FGF2-induced FGFR4 R388 phosphorylation and FGFR1 downregulation (FIG. 7).

This Example establishes that certain anti-FGFR4 antibodies block invasion of cancer cells that express both MT1-MMP and FGFR4 R388.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

96. An isolated monoclonal antibody, or fragment thereof, or a polypeptide that comprises a fragment of the antibody;
wherein the antibody, fragment, or polypeptide binds an extracellular epitope of a fibroblast growth factor receptor-4 (FGFR4) that is expressed by mammalian cells; and
wherein the antibody, fragment, or polypeptide exhibits at least one activity selected from the group consisting of:
inhibiting cancer cell invasion in a mammal;
inhibiting fibroblast growth factor 2 (FGF2)-induced phosphorylation of FGFR4 R388 in mammalian cells that express FGFR4 R388;
enhancing FGF2-induced degradation of fibroblast growth factor receptor-1 (FGFR1) in mammalian cells that co-express FGFR4 R388 and FGFR1; and
inhibiting complex formation between FGFR4 and membrane type-1 metalloproteinase (MT1-MMP) in mammalian cells that co-express FGFR4 R388 and MT1-MMP.

97. The isolated monoclonal antibody, antibody fragment, or polypeptide according to claim 96 that binds an extracellular epitope of an FGFR4 that comprises the amino acid sequence of SEQ ID NO: 1 or 2.

98. The isolated monoclonal antibody, antibody fragment, or polypeptide of claim 97, wherein the antibody, fragment, or polypeptide binds at least one FGFR4 peptide that consists of an amino acid sequence selected from the group consisting of SEQ ID NOS: 5-9.

99. The isolated monoclonal antibody, antibody fragment, or polypeptide of claim 97, wherein the antibody, fragment, or polypeptide binds an FGFR4 extracellular epitope that comprises amino acid residues 79-81 of SEQ ID NO: 1 or 2.

100. The isolated monoclonal antibody, antibody fragment, or polypeptide of claim 97, wherein the antibody is monoclonal antibody F90-10C5 (DSM ACC2967).

101. The isolated monoclonal antibody, antibody fragment, or polypeptide of claim 97 that is a humanized antibody, a human antibody, a chimeric antibody, or comprises a fragment of the human, humanized, or chimeric antibody that binds an extracellular epitope of FGFR4 that is expressed by mammalian cells.

102. The isolated monoclonal antibody, antibody fragment, or polypeptide of claim 101, further comprising an anti-neoplastic or cytotoxic agent conjugated or attached thereto.

103. A composition comprising the isolated monoclonal antibody, antibody fragment, or polypeptide of claim 101 (“the first monoclonal antibody or fragment thereof”) and a physiologically acceptable carrier.

104. The composition of claim 103, further comprising a second monoclonal antibody or fragment thereof, or a polypeptide that comprises a fragment thereof (“second monoclonal antibody or fragment thereof”), wherein the second monoclonal antibody or fragment thereof binds a second extracellular epitope of the FGFR4 that is different than the epitope recognized by the first monoclonal antibody or fragment thereof.

105. The composition of claims 103, further comprising a membrane type-1 metalloproteinase (MT1-MMP) inhibitor.

106. An isolated monoclonal antibody, or fragment thereof, or a polypeptide that comprises a fragment of the antibody; wherein the antibody, fragment, or polypeptide binds an FGFR4 extracellular epitope that comprises amino acid residues 79-81 of SEQ ID NO: 1 or 2.

107. An isolated cell that produces the antibody, antibody fragment, or polypeptide of claim 96.

108. The isolated cell of claim 107 that is from a hybridoma cell line deposited under Deutsche Sammlung von Mikroorganismen and Zellkulturen GmbH (DSMZ) Deposit Accession Number DSM ACC2967.

109. An isolated monoclonal antibody, antibody fragment, or polypeptide that comprises a fragment of the antibody, wherein the antibody, fragment, or polypeptide comprises all complementarity determining regions (CDR) of monoclonal antibody F85-6C5 (DSM ACC2966) or monoclonal antibody F90-3B6 (DSM ACC2965), or comprises the variable regions of monoclonal antibody F85-6C5 or F90-3B6, and wherein the antibody, antibody fragment, or polypeptide binds an extracellular epitope of FGFR4 that is expressed by mammalian cells.

110. An isolated cell that produces the antibody, antibody fragment, or polypeptide of claim 109.

111. The isolated cell of claim 110 that is from a hybridoma cell line selected from: hybridoma cell line deposited under DSMZ Deposit Accession Number DSM ACC2966; and hybridoma cell line deposited under DSMZ Deposit Accession Number DSM ACC2965.

112. A method of treatment comprising: administering to a mammalian subject with cancer the antibody, antibody fragment, or polypeptide of claim 96.

113. The method of claim 112, wherein the antibody, antibody fragment, or polypeptide is administered in an amount effective to inhibit invasion, ingrowth, or metastasis of cancer cells in the mammalian subject.

114. The method of claim 112, wherein the mammalian subject is human.

115. The method of claim 114, wherein the cancer is selected from breast cancer, bladder cancer, melanoma, prostate cancer, mesothelioma, lung cancer, testicular cancer, thyroid cancer, squamous cell carcinoma, glioblastoma, neuroblastoma, uterine cancer, colorectal cancer, and pancreatic cancer.

116. The method according to claim 114, wherein the cancer is a cancer determined to have at least one FGFR4 allele that encodes FGFR4 R388.

117. The method of claim 114, further comprising a step of determining the presence or absence of an FGFR4 allele that encodes FGFR4 R388 in the cancer, wherein the treatment is administered if the cancer has at least one FGFR4 allele that encodes FGFR4 R388.

118. The method according to claim 114, further comprising administering to the subject one or more of the following:
a second monoclonal antibody or fragment thereof, wherein the second monoclonal antibody or fragment thereof binds a second extracellular epitope of FGFR4 that is different than the epitope recognized by the first monoclonal antibody or fragment thereof;
a composition comprising a membrane type-1 metalloproteinase (MT1-MMP) inhibitor; and
a standard of care anti-cancer therapy.

119. The method according to claim 118, comprising administering the second monoclonal antibody or fragment thereof, wherein the second monoclonal antibody or fragment inhibits ligand-independent FGFR4 phosphorylation.

120. A composition comprising an adjuvant and at least one of:
an isolated antigenic peptide consisting of 5-25 amino acids of the amino acid sequence encoding FGFR4, wherein the peptide comprises the amino acid sequence set forth in any one of SEQ ID NOs: 5-9 or a fragment thereof;
an isolated polynucleotide encoding the antigenic peptide; and
a vector comprising the polynucleotide.

121. A method of selecting an antibody or antibody fragment, wherein the method comprises:
(a) obtaining one or more antibodies or antibody fragments that bind FGFR4;
(b) screening the antibodies or antibody fragments in a tumor cell invasiveness assay; and
(c) selecting an antibody that inhibits invasiveness in the assay by at least 50%.


SEQUENCE LISTING (18 sequences)

SEQ ID NO: 1 - protein, 802 aa, Homo sapiens [sequence omitted]

SEQ ID NO: 2 - protein, 802 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGAVGFPYVQVLKTADINSSEVEVLYLRNVSAEDAGEYTCLAGNSIGLSYQSAWLTVLPEEDPTWTAAAPEARYTDIILYASGSLALAVLLLLARLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 3 - protein, 25 aa, Homo sapiens
RYTDIILYASGSLALAVLLLLAGLY

SEQ ID NO: 4 - protein, 25 aa, Homo sapiens
RYTDIILYASGSLALAVLLLLARLY

SEQ ID NO: 5 - protein, 15 aa, Homo sapiens
YKEGSRLAPAGRVRG

SEQ ID NO: 6 - protein, 15 aa, Homo sapiens
GSRLAPAGRVRGWRG

SEQ ID NO: 7 - protein, 15 aa, Homo sapiens
LAPAGRVRGWRGRLE

SEQ ID NO: 8 - protein, 15 aa, Homo sapiens
AGRVRGWRGRLEIAS

SEQ ID NO: 9 - protein, 15 aa, Homo sapiens
VRGWRGRLEIASFLP

SEQ ID NO: 10 - protein, 582 aa, Homo sapiens [sequence omitted]

SEQ ID NO: 11 - DNA, 2915 bp, Homo sapiens
tggggcggggctgttgggaagtccagcttgggtccctgagagctgtgagaaggagatgcggctgctgctggccctgttgggggtcctgctgagtgtgcctgggcctccagtcttgtccctggaggcctctgaggaagtggagcttgagccctgcctggctcccagcctggagcagcaagagcaggagctgacagtagcccttgggcagcctgtgcgtctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacccctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgctataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgtcggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccaggtatacggacatcatcctgtacgcgtcgggctccctggccttggctgtgctcctgctgctggccgggctgtatcgagggcaggcgctccacggccggcacccccgcccgcccgccactgtgcagaagctctcccgcttccctctggcccgacagttctccctggagtcaggctcttccggcaagtcaagctcatccctggtacgaggcgtgcgtctctcctccagcggccccgccttgctcgccggcctcgtgagtctagatctacctctcgacccactatgggagttcccccgggacaggctggtgcttgggaagcccctaggcgagggctgctttggccaggtagtacgtgcagaggcctttggcatggaccctgcccggcctgaccaagccagcactgtggccgtcaagatgctcaaagacaacgcctctgacaaggacctggccgacctggtctcggagatggaggtgatgaagctgatcggccgacacaagaacatcatcaacctgcttggtgtctgcacccaggaagggcccctgtacgtgatcgtggagtgcgccgccaagggaaacctgcgggagttcctgcgggcccggcgccccccaggccccgacctcagccccgacggtcctcggagcagtgaggggccgctctccttcccagtcctggtctcctgcgcctaccaggtggcccgaggcatgcagtatctggagtcccggaagtgtatccaccgggacctggctgcccgcaatgtgctggtgactgaggacaatgtgatgaagattgctgactttgggctggcccgcggcgtccaccacattgactactataagaaaaccagcaacggccgcctgcctgtgaagtggatggcgcccgaggccttgtttgaccgggtgtacacacaccagagtgacgtgtggtcttttgggatcctgctatgggagatcttcaccctcgggggctccccgtatcctggcatcccggtggaggagctgttctcgctgctgcgggagggacatcggatggaccgacccccacactgccccccagagctgtacgggctgatgcgtgagtgctggcacgcagcgccctcccagaggcctaccttcaagcagctggtggaggcgctggacaaggtcctgctggccgtctctgaggagtacctcgacctccgcctgaccttcggaccctattccccctctggtggggacgccagcagcacctgctcctccagcgattctgtcttcagccacgaccccctgccattgggatccagctccttccccttcgggtctggggtgcagacatgagcaaggctcaaggctgtgcaggcacataggctggtggccttgggccttggggctcagccacagcctgacacagtgctcgaccttgatagcatggggcccctggcccagagttgctgtgccgtgtccaagggccgtgcccttgcccttggagctgccgtgcctgtgtcctgatggcccaaatgtcagggttctgctcggcttcttggaccaaggcgcttagtccccatcccgggtttggctgagcctggctggagagctgctatgctaaacctcctgcctcccaataccagcaggaggttctgggcctctgaaccccctttccccacacctccccctgctgctgctgccccacgctcttgacgggagcattggcccctgagcccagagaagctggaagcctgccgaaaacaggagcaaatggcgttttataaattatttttttgaaataaaa

SEQ ID NO: 12 - DNA, 2915 bp, Homo sapiens
tggggcggggctgttgggaagtccagcttgggtccctgagagctgtgagaaggagatgcggctgctgctggccctgttgggggtcctgctgagtgtgcctgggcctccagtcttgtccctggaggcctctgaggaagtggagcttgagccctgcctggctcccagcctggagcagcaagagcaggagctgacagtagcccttgggcagcctgtgcgtctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacccctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgctataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgtcggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccaggtatacggacatcatcctgtacgcgtcgggctccctggccttggctgtgctcctgctgctggccaggctgtatcgagggcaggcgctccacggccggcacccccgcccgcccgccactgtgcagaagctctcccgcttccctctggcccgacagttctccctggagtcaggctcttccggcaagtcaagctcatccctggtacgaggcgtgcgtctctcctccagcggccccgccttgctcgccggcctcgtgagtctagatctacctctcgacccactatgggagttcccccgggacaggctggtgcttgggaagcccctaggcgagggctgctttggccaggtagtacgtgcagaggcctttggcatggaccctgcccggcctgaccaagccagcactgtggccgtcaagatgctcaaagacaacgcctctgacaaggacctggccgacctggtctcggagatggaggtgatgaagctgatcggccgacacaagaacatcatcaacctgcttggtgtctgcacccaggaagggcccctgtacgtgatcgtggagtgcgccgccaagggaaacctgcgggagttcctgcgggcccggcgccccccaggccccgacctcagccccgacggtcctcggagcagtgaggggccgctctccttcccagtcctggtctcctgcgcctaccaggtggcccgaggcatgcagtatctggagtcccggaagtgtatccaccgggacctggctgcccgcaatgtgctggtgactgaggacaatgtgatgaagattgctgactttgggctggcccgcggcgtccaccacattgactactataagaaaaccagcaacggccgcctgcctgtgaagtggatggcgcccgaggccttgtttgaccgggtgtacacacaccagagtgacgtgtggtcttttgggatcctgctatgggagatcttcaccctcgggggctccccgtatcctggcatcccggtggaggagctgttctcgctgctgcgggagggacatcggatggaccgacccccacactgccccccagagctgtacgggctgatgcgtgagtgctggcacgcagcgccctcccagaggcctaccttcaagcagctggtggaggcgctggacaaggtcctgctggccgtctctgaggagtacctcgacctccgcctgaccttcggaccctattccccctctggtggggacgccagcagcacctgctcctccagcgattctgtcttcagccacgaccccctgccattgggatccagctccttccccttcgggtctggggtgcagacatgagcaaggctcaaggctgtgcaggcacataggctggtggccttgggccttggggctcagccacagcctgacacagtgctcgaccttgatagcatggggcccctggcccagagttgctgtgccgtgtccaagggccgtgcccttgcccttggagctgccgtgcctgtgtcctgatggcccaaatgtcagggttctgctcggcttcttggaccaaggcgcttagtccccatcccgggtttggctgagcctggctggagagctgctatgctaaacctcctgcctcccaataccagcaggaggttctgggcctctgaaccccctttccccacacctccccctgctgctgctgccccacgctcttgacgggagcattggcccctgagcccagagaagctggaagcctgccgaaaacaggagcaaatggcgttttataaattatttttttgaaataaaa

SEQ ID NO: 13 - DNA, 2369 bp, Homo sapiens
agttcagtgcctaccgaagacaaaggcgccccgagggagtggcggtgcgaccccagggcgtgggcccggccgcggagcccacactgcccggctgacccggtggtctcggaccatgtctcccgccccaagaccctcccgttgtctcctgctccccctgctcacgctcggcaccgcgctcgcctccctcggctcggcccaaagcagcagcttcagccccgaagcctggctacagcaatatggctacctgcctcccggggacctacgtacccacacacagcgctcaccccagtcactctcagcggccatcgctgccatgcagaagttttacggcttgcaagtaacaggcaaagctgatgcagacaccatgaaggccatgaggcgcccccgatgtggtgttccagacaagtttggggctgagatcaaggccaatgttcgaaggaagcgctacgccatccagggtctcaaatggcaacataatgaaatcactttctgcatccagaattacacccccaaggtgggcgagtatgccacatacgaggccattcgcaaggcgttccgcgtgtgggagagtgccacaccactgcgcttccgcgaggtgccctatgcctacatccgtgagggccatgagaagcaggccgacatcatgatcttctttgccgagggcttccatggcgacagcacgcccttcgatggtgagggcggcttcctggcccatgcctacttcccaggccccaacattggaggagacacccactttgactctgccgagccttggactgtcaggaatgaggatctgaatggaaatgacatcttcctggtggctgtgcacgagctgggccatgccctggggctcgagcattccagtgacccctcggccatcatggcacccttttaccagtggatggacacggagaattttgtgctgcccgatgatgaccgccggggcatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaaggagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagattgatgctgctctcttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagccagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcttcttcagacgccatgggacccccaggcgactgctctactgccagcgttccctgctggacaaggtctgacgcccaccgccggcccgcccactcctaccacaaggactttgcctctgaaggccagtggcagcaggtggtggtgggtgggctgttcccatcgtcccgagccccctccccgcagcctccttgcttctctctgtcccctggctggcctccttcaccctgaccgcctccctccctcctgccccggcattgcatcttccctagataggtcccctgagggctgagtgggagggcggccctttccagcctctgcccctcaggggaaccctgtagctttgtgtctgtccagccccatctgaatgtgttgggggctctgcacttgaaggcaggaccctcagacctcgctggtaaaggtcaaatggggtcatctgctccttttccatcccctgacataccttaacctctgaactctgacctcaggaggctctgggcactccagccctgaaagccccaggtgtacccaattggcagcctctcactactctttctggctaaaaggaatctaatcttgttg

SEQ ID NO: 14 - DNA, 21 bp, Homo sapiens
cagcgatgaagtcttcactta

SEQ ID NO: 15 - DNA, 21 bp, Homo sapiens
ccgcctgaccttcggacccta

SEQ ID NO: 16 - DNA, 21 bp, Homo sapiens
cacattgactactataagaaa

SEQ ID NO: 17 - DNA, 18 bp, Homo sapiens
taccagtctgcctggctc

SEQ ID NO: 18 - DNA, 18 bp, Homo sapiens
agtacgtgcagaggcctt